(19) United States
(12) Patent Application Publication
(10) Pub. No.: US 2021/0000920 A1
(43) Pub. Date: Jan. 7, 2021

(54) INTRADUCTAL METHODS OF TREATMENT OF BREAST DISORDERS

(71) Applicant: Atossa Therapeutics, Inc., Seattle, WA (US)

(72) Inventor: Steven C. Quay, Seattle, WA (US)

(73) Assignee: Atossa Therapeutics, Inc., Seattle, WA (US)

(21) Appl. No.: 16/956,949

(22) PCT Filed: Dec. 20, 2018
(86) PCT No.: PCT/US2018/066860

§ 371 Date: Jun. 22, 2020

Related U.S. Application Data
(60) Provisional application No. 62/609,665, filed on Dec. 22, 2017.

(51) Int. Cl. A61K 38/20; A61K 9/51; A61K 9/00

(52) U.S. Cl. CPC A61K 38/208; A61K 45/06; A61K 9/0041; A61K 9/5123

(57) ABSTRACT

The present invention relates to intraductal methods and compositions for treating subjects having breast disorders. Compositions comprise repolarizing agents and polarization blockading agents capable of repolarizing M2-macrophages to M1-macrophages in the tumor microenvironment, decreasing M2-macrophages, increasing M1-macrophages and/or increasing sensitivity to chemotherapy in the subject.

### CROSS-REFERENCE TO RELATED APPLICATION

This application claims the benefit of U.S. Application No. 62/609,665, filed Dec. 22, 2017, which is incorporated herein by reference in its entirety.

### BACKGROUND

Breast cancer is the second leading cause of cancer death in humans. Despite advances in diagnosing and treating breast cancer, the prevalence of this disease has been steadily rising at a rate of about 1% per year since 1940. Today, the likelihood that a woman living in North America will develop breast cancer during her lifetime is one in eight. Despite recent advances in early diagnosis and treatment that has significantly improved outcomes, tumor resistance to chemotherapy and metastasis remains significant causes of mortality.

The presence of hostile tumor microenvironment (TME) has resulted in poor access of the administered drugs to target cells and in tumor escape. The TME includes conditions such as high tissue pressure, hypoxia, nutritional starvation (for example, due to reduced glucose and other metabolites), increased lactate generation and resulting acidosis, increased infiltration of regulatory CD4+ T-cells (T-regs), myeloid derived suppressor cells (MDSCs), and tumor associated macrophages (TAMs), presence of immunosuppressive cytokines (such as IL-10 and TGF-β), and expression of ligands targeted to immune suppressive receptors expressed by activated T-cells (such as CTLA4 and PD-1), and reduced T-cell proliferation, which helps to create immunosuppressive microenvironment allowing tumors to protect themselves from immune recognition, maintain tolerance, and evade elimination.

Tumor microenvironment (TME) is a complex structure that evolves with tumor progression to promote metastatic spread. In solid tumors, 5%-40% of the tumor mass consists of TAMs. TAMs can represent the most abundant immunosuppressive cell population in the tumor microenvironment, recruited by CSF-1 and CCL-2 (MCP-1) (Sica et al. 2006, Eur J Cancer., 42, 717-727).

Resident macrophages in the breast are important for acting as sensors for tissue damage and maintain tissue homeostasis and immunity. Macrophages are critical mediators of inflammation and important regulators of mammary developmental processes. Balance between proinflammatory and anti-inflammatory factors is key to the regulation of macrophage function within the TME. During inflammation and tumor development, circulating monocytes are recruited to the site of inflammation and to TME where they adopt a macrophage phenotype dictated by the presence of specific cytokines and growth factors. Once recruited to the tumor microenvironment, macrophages respond to the plethora of stimuli within the TME and differentiate (polarize) into various subsets. In humans, macrophage polarization is a continuum that spans two extremes from the classically activated microbicidal and pro-inflammatory M1 macrophages to the alternatively activated anti-inflammatory and immunosuppressive M2 macrophages.

The M1 macrophages are polarized by interferon gamma (IFNγ) or lipopolysaccharide (LPS) stimuli, and secrete proinflammatory cytokines such as IL-6, IL-12, reactive oxygen species (ROS), reactive nitrogen (RN), and tumor necrosis factor alpha (TNFα). The validated cells surface markers of human M1 macrophages include high levels of CD14, C16, CD64, CD86, and HLA-DRα. M1 macrophages generally serve to eliminate pathogens and cells.

In contrast, M2 macrophages generally produce anti-inflammatory cytokines and are involved in tissue remodeling and repair, angiogenesis, immunosuppression, and recruitment of regulatory T-cells. M2 macrophages can be further divided into M2a, M2b, M2c macrophages. M2a macrophages arise from IL-4 or IL-13 stimuli and release matrix-remodeling cytokines. Elevated expression of CD200R and CD86 are validated cell surface markers of M2a macrophages. M2b macrophages results from the recognition of immune complexes in combination with IL-1β or LPS stimuli and like M2a macrophages, they are involved in wound healing. The immunosuppressive M2c macrophages are the outcome of IL-10, TGFβ, glucocorticoids, or immune complex rich environments. M2c macrophages generate further immunosuppressive cytokines such as IL-10 and matrix-remodeling factors such as matrix metalloproteases (MMPs; e.g., MMP-9). Elevated CD163 expression is a validated marker of M2c polarization.

TAM phenotypes are thought to include a combination of markers typically assigned to the M1 and M2 phenotypes based on transcriptomic studies on TAMs (J. Xue, S. V. Schmidt, J. Sander et al., Immunity, Vol. 40, no. 2, pp. 274-288, 2014). However, the TAMs are typically associated with M2-like polarization state caused by tumor-derived lactic acid or secretion of immunosuppressive cytokines such as IL-4, IL-10, and IL-13 from the different cells in the TME or B-cell derived immunoglobulins based on their function in the TME (Brady et al. Mediators of Inflammation. Volume 2016 (2016), Article ID 4549676).

Despite being generally anti-inflammatory, immunosuppressive and tumorigenic, attempts are being made to make TAMs tumoricidal and suppress tumor growth by activating immune response (Wynn et al. Nature 2013, 496:445-455). A low ratio of macrophages to CD8+ cells in breast cancers predicts survival suggesting a major role for macrophages in suppressing T cell activity against tumors (Ruffell, B. et al. P.N.A.S. USA. 2012; 109, 2796-2801). Infiltration of tumors by specific leukocyte cell subsets such as CD8+ and memory T-cells have linked to favorable outcomes in different cancers, suggesting that immune engagement can limit cancer growth and spread (Noy R, Pollard J W. Immunity, 2014; 41:49-61). Enhanced T-cell activity in tumors using checkpoint inhibitors such as anti-PD-1 has been found to be successful to some extent in clinical trials treating melanoma and lung cancers.

Reprogramming or educating of TAMs by repolarizing the TAMs to M1 activated macrophages are being studied, for example, by systemically delivered antibody-mediated co-stimulation of CD40, Il-12 cytokine, and the like (Casetta L, Pollard J W. Cell Research (2017) 27:963-964); Georgoudaki et al. Cell Reports. 2016. 15, 2000-2011; Guerriero et al. Nature 2017. 543;428-432; U.S. Pat. Nos. 9,139,652; 9,795,570; US20170266131; US20170056430; US20160220692; US20150297679; US20150252115; and US20150080940), there remains an unmet medical need for novel strategies for the treatment of breast disorders such as breast cancer and the maintenance of macrophage homeostasis. The present invention provides novels methods for treating subject with breast disorders, improving sensitivity to chemotherapy, and promoting macrophage homeostasis.

### SUMMARY OF THE INVENTION

It is an object of the present invention to provide novel methods of treating a subject having a breast disorder, the method comprising delivering intraductally to the subject a composition comprising a repolarizing agent capable of repolarizing a M2-polarized macrophage, or a blockading agent capable of blocking M2 polarization of macrophages, or both.

In another aspect, the present invention provides novel methods of promoting increased sensitivity to chemotherapy in a subject having a breast disorder, comprising: administering intraductally to the subject a composition comprising a repolarizing agent or a blockading agent or both, wherein intraductal administration of the composition promotes increased sensitivity to chemotherapy.

In at least one embodiment, the present invention provides methods for selective reduction of M2 macrophages in a subject having a breast disorder comprising intraductally administering to the subject a composition comprising a repolarizing agent or a blockading agent or both.

Intraductal administration of the composition results in one or more of: decreased M2 macrophages, increased M1 macrophages, decreased anti-inflammatory cytokines, increased pro-inflammatory cytokines, recruitment of cytotoxic T-lymphocytes and T-lymphocyte activation.

In some embodiments, the M2 macrophage phenotype is any one or more of M2a phenotype, M2b phenotype, and M2c phenotype, or a combination thereof.

In an aspect, the present invention provides that intraductal administration of compositions disclosed herein results in a decrease in any one or more of the macrophage population selected from the group consisting of: F4/80+ macrophages; Grl− macrophages; CD206+ macrophages; CD200R cells/macrophages; CD63+ macrophages; CD68+/CD68+ macrophages; CD68+/CD163+ macrophages; MARCO+ macrophages; Tie2R+ cells/macrophages; CD11b+/VEGF-R1+ macrophages; CCR2+ myeloid cells/macrophages; MerTK+ macrophages; CD11b^{low}/MHCII^{high}/CCR2+/F4/80+/CD64+/MerTK+ macrophages; CD11b+Grl−/F4/80 +macrophages; CD45+/CD11b+/Ly6G−/Ly6C^{low}/F4/80+ macrophages; CD11b+/F4/80+/MHCII+/Ly6C− macrophages; CD45+/CD11b+/F4/80+/Tie2+/CD31− macrophages; and CD45+/F480+/Tie2−/CD31− macrophages, or a combination thereof.

In another aspect, the present invention provides that intraductal administration of compositions disclosed herein results in an increase in any one or more of the macrophage population selected from the group consisting of: CD11b^{high}/MHCII^{high} macrophages; HLA-DRα+ macrophages; CD86+ macrophages; CD80+ macrophages; CD68+/CD80+ macrophages; CD64+ macrophages; and Ly6C^{high}/CX3CR1^{high}/CCR2−/CD62L−/CD43^{low} (Ly6C^{high}) macrophages, or a combination thereof.

In one aspect, the present invention provides repolarizing agents selected from the group consisting of fenretinide (4-hydroxy(phenyl)retinamide, 4-HPR); IL-12; IFNγ; miR127; miR155; miR223; ferumoxytol; CD40L; inhibitors of: CSF-1, CSF-1R, IL-10, IL-10R, TGFβ, Arginase 1 (Arg1), M2 macrophage scavenger receptors (such as A, B, MARCO), histone deacetylase (HDACi), DICER, IRF4/STAT4/STAT6 signaling pathway, IL-4, IL-13, IL-17, PPARγ, KLF4, KLF6, miRNA-146 family members such as (miRNA-146a), let7 family members (such as let-7c), miRNA-9, miRNA-21, miRNA-47, miRNA-187; CCR-CCl2 axis signaling, CCL2/MCP-1 synthesis, placental growth factor (PlGF) (HRG) and C/EBPβ (PI3Kγ deletion), AMPKα1 (metformin), p50-p50 NFκB, NADPH oxidase (NOX) (NOX 1 and NOX 2), Notch signaling pathway and Rbpj; activators of: CD40, CD40L, IRF1, IRF5, STAT1 (such as IFNγ, vadimezan (DMXAA)), STAT3, nuclear factor kappa B activators, toll-like receptor (TLR) agonists such as Imiquimod, synthetic unmethylated cytosine-guanine (CpG) oligodeoxinucleotides (CpG-ODNs), p65-p50 NFκB, MyD88, miR127, miR155, and miR223, or a combination thereof.

In one aspect, the present invention provides blockading agents selected from the group consisting of anti-CSF-1 inhibitors, anti-CSF-1R inhibitors, anti-MCP-1 inhibitors, anti-IL-4 inhibitors (such as pascolizumab, pitakinra and dupilumab), anti-IL-13 inhibitors (such as anrukinzumab, lebrikizunab and tralokinumab), anti-IL-4/IL-13 dual inhibitors such as duplimab, STAT3 inhibitors (such as sorafenib, sunitinib, WP1066, and resveratrol), and STAT6 inhibitors (such as fenretinide (4-HPR), leflunomid, TMX264, and AS1217499), or a combination thereof.

In some embodiments, one or more HDACi is selected from the group consisting of TMP195, MC1568, TMP269, an (aryloxopropenyl)pyrrolyl hydroxamate), trichostatin A, trapoxin B, tubastatin A hydrochloride (anti-HDAC7), Panobinostat, suberoylanilide hydroxamic acid (SAHA) a Class I and Class II inhibitor Vorinostat (Volinza® Merck), Romidepsin (Istodax®); Depsipeptide, FK-228), Belinostat (PXD-101), Panobinostat (LBH589), Dacinostat (LAQ824), SB939, Chidamide, pan-HDAC inhibitors (such as Givinostat (ITF2357), PCI 2478, R306465 (JNJ-16241199), Resminostat (4SC-201)), valproic acid, butyric acid, phenylbutyrate, AN9/Pivanex, Class I-selective HDAC inhibitors benzamides or amino analides (such as CI-994, Entinostat (SNDX-275/MS-275), Mocetinostat (MGCD0103), Abexinostat (PCI-24781), Quisinostat (JNJ-26481585), HBI-8000, Kevetrin, CUDC-101, AR-42, CHR-2845, CHR-3996, 4SC-202, CG200745, ACY-1215, ME-344, and Sulforaphane, or a combination thereof.

In other embodiments, the MARCO inhibitor is selected from the group consisting of anti-MARCO antibodies (such as ab103311, monoclonal ED31, PLK-1, ABN 1389), anti-MARCO ScFv, Fab, Fab′, and Fab2, anti-MARCO ScFv mRNA, anti-MARCO miRNA, MARCO anti-sense RNA, MARCO siRNA, anti-MARCO DNA, anti-MARCO oligonucleotides, anti-MARCO peptide inhibitors, or a combination thereof.

In some embodiments, the composition disclosed herein further comprises a pharmaceutically acceptable carrier.

In an aspect, the present invention provides that compositions further comprise an additional therapeutic agent. In some embodiments, the additional therapeutic agent is selected from the group consisting of checkpoint inhibitors, anti-hormonals, steroids, anthracyclines, thyroid hormone replacement drugs, thymidylate-targeted drugs (such as docetaxel, gemcitabine, paclitaxel or carboplatin and pegylated liposomal doxorubicin), DNA hypomethylating agents (such as azacitidine or decitabine), trastuzumab, ado-trastuzumab emtansine, pertuzumab, abemaciclib, palbociclib, cell therapy such as Chimeric Antigen Receptor/T cell (CAR-T) therapies, and other adoptive cell therapies, or a combination thereof.

In some embodiments, the anti-hormonal is selected from the group consisting of tamoxifen, cis-tamoxifen, endoxifen, desmethyltamoxifen, lasofoxifene, raloxifene, benzothiophene, bazedofoxifene, arzoxifene, miproxifene, levormeloxifene, droloxifene, clomifene, idoxifene, toremifene, EM652 and ERA-923, fulvestrant, ARN-810, or CH498, anastrozole, exemestane and letrozole, or a combination thereof.

In some embodiments, the checkpoint point inhibitor is selected from the group consisting of anti-PD-1 (such as Nivolumab), anti-PD-1L (such as atezolizumab (MPDL3280), Avelumab (MSB0010718C), Durvalumab, MDX-1105), anti-CTLA4 (e.g., Ipilimumab), and anti-LAG-3 (such as IMP321, BMS-986016 and GSK2831781), or a combination thereof.

In another aspect, the present invention provides that the compositions disclosed herein further comprise an imaging agent, a dye or a contrasting agent selected from the groups consisting of gadolinium chelates, superparamagnetic iron oxide nanoparticles (SPION), 19F perfluorocarbon nanoparticles, and other magnetic reporter genes, such as metalloprotein-based MRI probes.

In one aspect, the present invention provides compositions comprising repolarizing agent or blockading agent or both formulated as liposomes, microparticles, microspheres, nanocapsules, nanoparticles, nanospheres, lipid particles, vesicles, micelles, and exosomes. In some embodiments, the repolarizing or the blockading agent or both are comprised in liposomes, microparticles, microspheres, nanocapsules, nanoparticles, nanospheres, lipid particles, vesicles, micelles, and exosomes. In other embodiments, the repolarizing or the blockading agent or both are comprised on the liposomes, microparticles, microspheres, nanocapsules, nanoparticles, nanospheres, lipid particles, vesicles, and micelles.

In at least one embodiment, the nanoparticle is a lipid nanoparticle. In some embodiments, the nanoparticle is coated with a cell targeting agent. The cell targeting agent is a M2-macrophage selective cell surface molecule. In certain embodiments, the M2-macrophage selective cell surface molecule is selected from the group consisting of IL-13Rα, CD163, CD206, CD200R, MerTK, scavenger receptor A, scavenger receptor B, MARCO, and F4/80. In some embodiments, the composition of any of the preceding claims, wherein the composition is formulated as a depot formulation.

In an aspect, the present disclosure provides that the subject is administered intraductally a composition comprising repolarizing agent or the blockading agent or both, wherein the composition comprises 1×10⁴ to 1×10⁸ liposomes, microparticles, microspheres, nanocapsules, nanoparticles, nanospheres, lipid particles, vesicles, or exosomes per unit dose. The subject may be administered the compositions disclosed herein in a single dose or multiple doses.

In an aspect, the breast disorder is breast cancer. In some embodiments, the breast cancer is a breast cancer selected from the group consisting of ductal carcinoma in situ (DCIS), lobular carcinoma in situ (LCIS), invasive (or infiltrating) lobular carcinoma (ILC), invasive (or infiltrating) ductal carcinoma (IDC), microinvasive breast carcinoma (MIC), inflammatory breast cancer, ER-positive (ER+) breast cancer, progesterone receptor positive (PR+) breast cancer, ER+/PR+ breast cancer, ER-negative (ER−) breast cancer, HER2+ breast cancer, triple negative breast cancer (i.e., ER−/PR−/Her2− breast cancer; “TNBC”), adenoid cystic (adenocystic) carcinoma, low-grade adenosquamatous carcinoma, medullary carcinoma, mucinous (or colloid) carcinoma, papillary carcinoma, tubular carcinoma, metaplastic carcinoma, or micropapillary carcinoma.

In some embodiments, the methods further comprise administering chemotherapy, radiotherapy, or cell therapy to the subject. In some embodiments, the chemotherapy, radiotherapy, or cell therapy or a combination thereof, reduces tumor size or immunosuppression or both, in the subject.

In other embodiments, the intraductal administration of a composition comprising a repolarizing agent or a blockading agent or both, increases in the subject any one or more of: tumoricidal activity of M1-polarized macrophages; release of pro-inflammatory cytokines, chemokines and growth factors; cytotoxic T-lymphocyte recruitment; and T-cell activation.

In still other embodiments, the intraductal administration of a repolarizing agent or a blockading agent or both, reduces in the subject any one or more of: tumor angiogenesis; tumor invasion; metastasis; immunosuppression; and release of anti-inflammatory cytokines, chemokines and growth factors.

In another aspect, the present invention provides an article of manufacture, comprising a composition comprising a repolarizing agent or a blockading agent or both, one or more containers, packaging material, a label or package insert, and optionally, a device. In some embodiments, the device is a needle and syringe, a cannula, a catheter, a microcatheter, an osmotic pump, or an encapsulation device. In other embodiments, the article of manufacture further comprises an additional therapeutic agent such as those disclosed herein.

### DETAILED DESCRIPTION

In general, disclosed herein are compositions that can be used to regulate macrophage activation, repolarize M2-macrophages in the TME and improve sensitivity to therapy such as chemotherapy, radiotherapy, immunotherapy, and cells therapy. Such compositions comprise repolarizing agents and polarization blockading agents capable of repolarizing M2-macrophages to M1-macrophages or blocking polarization of infiltrating monocytes and macrophages to M2-macrophages. Also, provided herein are methods of treatment of subjects having a breast disorder by administering compositions disclosed herein intraductally to one or more breast ducts of the subject.

### Macrophages and TAMs—Polarization and Phenotype

Resident macrophages (CD11b^{high}/MHCII^{high}) in the breast are important for acting as sensors for tissue damage and maintain tissue homeostasis and immunity. Macrophages are critical mediators of inflammation and important regulators of mammary developmental processes. Balance between proinflammatory and anti-inflammatory factors is key to the regulation of macrophage function within the TME. TME releases various cytokines, chemokines and growth factors to recruit circulating monocyte to the TME wherein they mature to form macrophages. Once recruited to the tumor microenvironment, macrophages respond to the plethora of stimuli within the TME and differentiate into various subsets. In humans, macrophage polarization is a continuum that spans two extremes from the classically activated pro-inflammatory M1 macrophages to the alternatively activated anti-inflammatory M2 macrophages.

Polarization of macrophages to the M1 phenotype is regulated in vitro by inflammatory signals such as GM-CSF, IFNγ, TNFα, IL-1β and LPS as well as various transcription factors and microRNAs (miRNAs). Interferon regulatory factors determine the phenotype of macrophage polarization. IFNγ polarizes macrophages to M1 phenotype via the interferon regulatory pathway using Interferon regulatory factor 1 (IRF1) and STAT1. LPS induces M1 phenotype via the Toll-like Receptor family (such as TLR-4). miRNAs are small non-coding RNA of 22 nucleotides in length that regulate gene expression post-transcriptionally, as they affect the rate of mRNA degradation. Several miRNAs have been shown to be highly expressed in M1-polarized macrophages, especially miRNA-155, miRNA-125, miRNA-127, miRNA-17, miRNA-20, miRNA-106a, and miRNA-378. Inhibition of these miRNA are said to reduce M1-polarization.

Classically activated macrophages initiate the induction of the STAT1 transcription factor which targets CXCL9, CXCL10 (also known as IP-10), IFN regulatory factor-1, and suppressor of cytokine signaling-1 (SOCS1). M1 macrophages secrete proinflammatory cytokines such as IL-6, IL-12, IL-23, reactive oxygen species (ROS) produced by the activity of enzymes NADPH oxidases NOX1 and NOX2, reactive nitrogen species (RN), and tumor necrosis factor alpha (TNFα) killing cells. M1 macrophages are highly phagocytic generally serving to eliminate pathogens and cells.

IL-12 induces the activation and clonal expansion of Th17 cells, which secrete high amounts of IL-17, which further contributes to inflammation. These characteristics allow M1 macrophages to control metastasis, suppress tumor growth, and control microbial infections. Studies suggest that infiltration and recruitment of pro-inflammatory M1 macrophages to tumor sites correlates with a better prognosis and higher overall survival rates in patients with solid tumors.

The validated cells surface markers of human M1 macrophages include high levels of CD14, C16, CD64, CD86, and HLA-DRα.

In contrast to M1 macrophages, M2 macrophages generally produce anti-inflammatory cytokines, and induce tissue remodeling and repair, angiogenesis, immunosuppression, and recruitment of regulatory T-cells. M2 macrophages can be further divided into M2a, M2b, M2c macrophages. M2a macrophages arise from IL-4 or IL-13 stimuli and release matrix-remodeling cytokines (Gunthner and Anders. Mediators of Inflammation. Vol. 2013 (2013), Article ID 731023). IL-4 polarizes macrophages to M2-phenotype using interferon regulatory pathway via IRF4 and STAT6 signaling. Elevated expression of CD200R and CD86 are validated cell surface markers of M2a macrophages. M2b macrophages results from the recognition of immune complexes in combination with IL-1β or LPS stimuli and like M2a macrophages, they are involved in wound healing. The immunosuppressive M2c macrophages are the outcome of IL-10, TGFβ, glucocorticoids, or immune complex rich environments. IL-10 polarizes macrophages to M2-phenotype using STAT3 signaling pathway (Gunthner and Anders. Mediators of Inflammation. Vol. 2013 (2013), Article ID 731023). M2c macrophages generate further immunosuppressive cytokines such as IL-10 and matrix-remodeling factors such as matrix metalloproteases (MMPs; e.g., MMP-9). Elevated CD163 expression, ARG1, RETNLB, IL4R, CHIA, CD68 are validated markers of M2c polarization.

Several miRNAs have been shown to be highly expressed in M2-polarized macrophages, especially miRNA-9, miRNA-21, miRNA147, miRNA-187, miRNA-146 family members such as (miRNA-146a), and let7 family members (such as let-7c). Inhibition of these miRNA are said to reduced M2-polarization. Studies have shown that deficiency of the miRNA-processing enzyme DICER results in the repolarization of TAMs to IFNγ-mediated M1-like phenotype characterized by enhanced expression of proinflammatory cytokines and T-cell—recruiting chemokines and that rescue with let-7 partially restores M2-macrophage phenotype (Squadrito and Palma. Cell Cycle. 2016, VOL. 15, NO. 23, 3149-3150). DICER inactivation in TAMs greatly enhanced the efficacy of cancer immunotherapies, namely PD1 checkpoint blockade and CD40 agonistic antibodies, leading to complete tumor regressions in mice. (Id.) Additional markers of M2-macrophages include, but are not limited to, Arginase 1 (Arg1), macrophage scavenger receptors A, B and MARCO, Tie2R+, Grl−, F4/80+, MerTK+, CD206+, and CD200R.

TAMs are thought to phenotypically include a combination of markers typically assigned to the M1-macrophage and M2-macrophage phenotypes based on transcriptomic studies on TAMs (Xue et al. Immunity. 2014. 40(2): 274-288). M2-like macrophage population subsets such as CD11b+Grl−/F4/80+ macrophages, CD45+/CD11b+/Ly6G−/Ly6Clow/F4/80+ macrophages, CD11b+/F4/80+/MHCII+/Ly6C− macrophages, CD45+/CD11b+/F4/80+/Tie2+/CD31− macrophages; and CD45+/F480+/Tie2−/CD31− macrophages have been found in breast cancers (Nielsen and Schmid. Mediators of Inflammation. Vol. 2017, Article ID. 9624760). However, based on their function in the TME, it is widely accepted that the TAMs are typically associated with M2-like polarization state caused by tumor-derived lactic acid or secretion of immunosuppressive cytokines such as IL-4, IL-10, and IL-13 from the different cells in the TME or B-cell derived immunoglobulins (Brady et al. Mediators of Inflammation. Vol. 2016 (2016), Article ID 4549676). For the purpose of the present disclosure, the present invention provides for repolarization of M2-like TAMs, including TAMs that may also show some M1-like phenotypic markers (“M2-macrophages” “M2-polarized macrophages” or “TAMs”, used interchangeably in this disclosure). M2-macrophage population, for the purpose of the present invention, may be any of the subsets of M2-macrophages (M2a, M2b, and M2c) or any combination thereof.

Heterogeneous TAM populations are found in distinct compartments within tumors based on the level of hypoxia in these areas. Inflammatory monocytes recruited to TME give rise to both MHCII^{low} and MHCII^{high} TAMs but TAMs inside hypoxic regions are predominantly MHCII^{low} and associated with increased expression of M2-markers. M2-like macrophage populations of TAM such as CD68+/CD68+ macrophages, CD68+/CD163+ macrophages, CD11b+/VEGF-R1+ macrophages, CCR2+ myeloid cells/macrophages, CD11b^{low}/MHCII^{high}/CCR2+/F4/80+/CD64+/MerTK+ macrophages, CD11b+Grl−/F4/80+ macrophages, CD45+/CD11b+/Ly6G−/Ly6Clow/F4/80+ macrophages, CD11b+/F4/80+/MHCII+/Ly6C− macrophages, CD45+/CD11b+/F4/80+/Tie2+/CD31− macrophages; and CD45+/F480+/Tie2−/CD31− macrophages have been found elevated in breast cancers (Nielsen and Schmid. Mediators of Inflammation. Vol. 2017, Article ID. 9624760).

### Methods of Treatment

In an aspect, provided herein are novel methods of treatment of a subject having a breast disorder, the method comprising delivering intraductally to a breast duct of the subject a composition comprising a repolarizing agent capable of repolarizing a M2-polarized macrophage, or a polarization blockading agent (“blockading agent” hereinafter) capable of blocking M2 polarization of macrophages, or both. Intraductal delivery in the methods disclosed herein is effected non-invasively or minimally-invasively, and typically does not involve breaking skin by a clinician delivering the treatment. Instead, the compositions are administered to a subject via the subject's own natural ductal orifice in the nipple of a mammary papilla.

As used herein, the terms “intraductal and “intraductally” refer to a method of treatment wherein the compositions disclosed herein are delivered into the lumen of at least one breast milk duct of a subject through the milk duct opening (ductal orifice) in the nipple on the mammary papilla(e) to reach the inner depths of the breast.

In one aspect, intraductal administration refers to the application of the compositions of the present disclosure to the nipple of a mammary papilla, wherefrom the compositions are delivered to at least one breast milk duct through a ductal orifice in the nipple. Breast nipples and ductal orifices are uniquely suited for receiving repolarizing agents, polarizing blockading agents, and therapeutic agents into ducts. In another aspect, intraductal administration refers to the intraductal delivery of compositions directly into the lumen of a breast milk duct via a ductal orifice in the nipple of a mammary papilla. It will be appreciated by a person of skill in the art that intraductal methods as disclosed herein comprise delivery of the compositions disclosed herein through a natural orifice of the breast milk duct in a subject's breast. An advantageous aspect of the present invention is that intraductal delivery typically does not involve deliberate breach of subject's skin or tissue or cell layer and delivers the compositions close to the affected breast tissue.

Breast disorders, such as breast cancers, typically originate in a milk duct of an individual (Wellings S R. Pathol. Res. Prac. 1980; 166:515-535; Love and Barsky. Cancer. 2004, vol 101(9):1947-1957). Thus, localized delivery of the therapy, for example, compositions comprising a repolarizing agent or a blockading agent, or both, close to the affected site within the breast milk ducts (breast ducts) is highly desirable. Intraductal administration of such compositions provide a local, effective, easy-to-administer therapy that would obviate the side effects of systemic treatment by reducing systemic exposure to the compositions comprising repolarizing agents and/or blockading agents. This would have the added benefit of reducing the possibility/risk of on-target-off-tumor effect. Local delivery to the breast duct by intraductal administration would reduce transit time, and provide faster exposure of the M2-macrophages in the TME to the compositions thereby, improving cytotoxic activity and efficacy while also reducing the potential for drug inactivation and/or degradation due to shorter transit to M2-macrophages. A particular advantage of the methods disclosed herein the in vivo transduction of macrophages in the breast tissue.

Intraductal administration of the compositions disclosed herein result in the appearance of one or more of: decreased M2 macrophages, increased M1 macrophages, decreased anti-inflammatory factors, increased pro-inflammatory factors, and increased recruitment of cytotoxic T lymphocytes. In some embodiments, the ratio of M2-macrophages to M1-macrophages is altered. The number of M1-macrophages are increased relative to M2-macrophages. In some embodiment, the repolarization restores M1/M2 macrophage homeostasis with regards to macrophage polarization status.

In some embodiments, the M2 macrophage phenotype that is repolarized is M2a phenotype, M2b phenotype, M2c phenotype, or a combination thereof. In some embodiments, the M2-like macrophage populations of TAM such as CD68+/CD163+ macrophages, CD11b+/VEGF-R1+ macrophages, CCR2+ myeloid cells/macrophages, CD68+/CD68+ macrophages, CD11b^{low}/MHCII^{high}/CCR2+/F4/80+/CD64+/MerTK+ macrophages, CD11b+Grl−/F4/80+ macrophages, CD45+/CD11b+/Ly6G−/Ly6Clow/F4/80+ macrophages, CD11b+/F4/80+/MHCII/Ly6C− macrophages, CD45+/CD11b+/F4/80+/Tie2+/CD31− macrophages; and CD45+/F480+/Tie2−/CD31− macrophages are repolarized by the compositions and methods disclosed herein. As a result, there is a decrease in the numbers of such M2-macrophages.

In some embodiments, the intraductal administration of the compositions disclosed herein results in the increase in the number of any one or more the macrophage population such as CD11b^{high}/MHCII^{high} macrophages, HLA-DRα+ macrophages, CD86+ macrophages, CD80+ macrophages, CD68+/CD80+ macrophage, CD64+ macrophages, or Ly6C^{high}/CX3CR1^{high}/CCR2−/CD62L−/CD43^{low} (Ly6C^{high}) macrophages.

In other embodiments, the intraductal administration of the compositions disclosed herein results in the increase of pro-inflammatory factors such as cytokines, chemokines and growth factors (for example, IL-1, IL-1β, IL-17, IFNγ, ROS, RNs, TNFα, GM-CSF), and the like. In other embodiments, the intraductal administration of the compositions results in decrease in the anti-inflammatory factors such as cytokines and chemokines (such as IL-4, IL-13, IL-10, CSF-1), growth factors (such as angiogenic growth factors like VEGF and TGFβ) and tissue-remodeling factors (such as MMPs such as MMP9), and the like. One of skill in the art will recognize that there are other known pro-inflammatory and anti-inflammatory factors that are suitable for the purpose of the present disclosure. The changes in the polarization/activation markers such as pro-inflammatory and immunosuppressive markers can be detected using any of the methods known in the art, for example, using ELISA, IHC, qPCR, and the transcriptome-based analysis described by Xue et al. in Immunity, vol. 40, no. 2, pp. 274-288, 2014.

Switching of M2-macrophage to M1-macrophage shifts the balance to a more pro-inflammatory state activating tumoricidal effector functions of the M1-macrophages. Accordingly, in some embodiments, the TAMs are repolarized from tumorigenic to tumoricidal macrophages. In still other embodiments, administration of compositions disclosed herein reduces the tumorigenic functions of M2-macrophages.

In some embodiments, the administration of a composition disclosed herein reduces tumor angiogenesis. M2-macrophages promote neo-angiogenesis to provide tumor tissue nourishment and aid in tumor metastasis by turning on the angiogenetic switch from a quiescent state in normal breast tissue to sprouting new blood vessels through the release of angiogenic growth factors such as vascular endothelial growth factor (VEGF-A) and placental growth factor (PlGF).

In other embodiments, the administration of a composition disclosed herein reduces tumor invasion. M2-macrophages release angiogenic growth factors such as VEGF-A that promote tumor cell intravasation into blood vessels. In addition, macrophages-derived cathepsins such as SPARC, or CCL8 enhance tumor cell adhesion to extracellular matrix proteins and promote tumor cell migration. M2-macrophages promote metastasis through their ability to engage tumor cells in an autocrine loop (for example, via CSF-1 and CSF-R production by tumor cells) that promotes increased infiltration of macrophages and tumor cell migration. Even where chemotherapy has eliminated macrometastatic lesions, a few drug resistant tumor cells service that eventually relapse as drug-resistant metastatic lesion. Studies in lungs have shown that ablation of CCR2+ macrophages reduce metastatic burden. Accordingly, in some embodiments, the administration of a composition disclosed herein reduces metastasis. In some embodiments, the administration of a composition disclosed herein reduces CCR2+ macrophages.

In further embodiments, the administration of a composition disclosed herein reduces immunosuppression. In still further embodiments, administration of compositions disclosed herein increases recruitment and activation of cytotoxic T-lymphocytes.

In at least one embodiment, the methods disclosed herein reduce the breast disease burden in the subject.

In another aspect, provided herein are novel methods for restoring or promoting macrophage homeostasis in the breast of a subject having a breast disorder comprising intraductal administration of a composition comprising a repolarizing agent or a blockading agent or both to one or more breast ducts of the subject.

In another aspect, provided herein are novel methods of promoting increased sensitivity to cancer therapy in a subject having a breast disorder, comprising: administering intraductally to the subject a composition comprising a repolarizing agent or a blockading agent or both. Macrophages play a key role in chemoresistance (Nielson and Schmid. Mediators of Inflammation. Vol. 2017. Article ID. 9624760). M2-macrophages in the TME induce the expression of the enzyme cytidine deaminase the primary metabolizing enzyme of the commonly used chemotherapeutic agent gemcitabine. Reprogramming the M2-macrophages to M1-macrophages using the intraductal methods disclosed herein increases the susceptibility of the tumor cells to chemotherapy and to increased T-cell and M1-macrophage mediated cytotoxicity.

In some embodiments, the methods comprise delivering chemotherapy, radiotherapy, immune therapy such as cell therapy (such as CAR-T therapy or universal T-cell therapy) to subjects. Increased sensitivity to cancer therapy results in increased tumor cell death. Tumor cell death in the subjects may be measured by any of the methods known in the art. For example, this can be detected by assays such as apoptosis detecting assay (TUNEL assay, R&D Systems) in tissue biopsy. Non-invasive methods of detecting tumor cell-death include measuring decreased lactate dehydrogenase that accompanies growth of tumors in a subject and/or decrease in ctDNA (circulating tumor-cell derived DNA) in blood samples (liquid biopsy) (Chang et al. Molecular Oncology, 2016; 10 (1): 157). Tumor cell-death in subjects may also be detected non-invasively using MR Imaging and a Gadolinium-based Targeted Contrast Agent (Krishnan et al. RSNA Radiology. March 2008, vol. 246, Issue 3), PET based measurement of uptake of the glucose analog, 2-¹⁸Fluoro-2-deoxy-glucose (¹⁸FDG), and hyperpolarized [1-13C]pyruvate using 13C magnetic resonance spectroscopy and spectroscopic imaging (Whitney et al. Proc. Intl. Soc. Mag. Reson. Med. 16 (2008); p658).

In another aspect, provided herein are novel methods for selective reduction of M2 macrophages in a subject having a breast disorder comprising intraductally administering to the subject an effective amount of the pharmaceutical composition comprising a reprogramming agent or a blockading agent or both.

As used herein, “breast disorder” means breast cancer. As used herein, “breast cancer” means any malignant tumor of breast cells. Breast cancer may be at any stage of breast cancer, including stages of a pre-cancer, an early stage cancer, a non-metastatic cancer, a pre-metastatic cancer, a locally advanced cancer, and a metastatic cancer. There are several types of breast cancer. Exemplary breast cancers include, but are not limited to, ductal carcinoma in situ (DCIS), lobular carcinoma in situ (LCIS), invasive (or infiltrating) lobular carcinoma (ILC), invasive (or infiltrating) ductal carcinoma (IDC), microinvasive breast carcinoma (MIC), inflammatory breast cancer, ER-positive (ER+) breast cancer, progesterone receptor positive (PR+) breast cancer, ER+/PR+ breast cancer, ER-negative (ER−) breast cancer, HER2+ breast cancer, triple negative breast cancer (i.e., ER−/PR−/Her2− breast cancer; “TNBC”), adenoid cystic (adenocystic) carcinoma, low-grade adenosquamatous carcinoma, medullary carcinoma, mucinous (or colloid) carcinoma, papillary carcinoma, tubular carcinoma, metaplastic carcinoma, or micropapillary carcinoma. A single breast cancer tumor can be any combination or a mixture of these types or be a mixture of invasive and in situ cancer.

DCIS is the most common non-invasive breast cancer. It involves the cell(s) lining the breast ducts. In DCIS, the cells have not spread beyond the walls of the duct into the surrounding breast tissue. About 1 in 5 new breast cancer cases will be DCIS. Several biomarkers are associated with DCIS. Exemplary biomarkers include, without limitation, estrogen receptor, progesterone receptor, androgen receptor, Ki-67, cyclin D1, cyclin A, cyclin E, p16, p21, p27, p53, Bcl-2, Bax, survivin, c-myc, Rb, VEGF, HPR1, HER1, HER2, HER3, HER4, CD10, SPARC, COX-2, basal cytokeratins, CK5/6, CK14, and CK17, epidermal growth factor receptor, Tn, ER+, and c-kit. DCIS is said to be a non-obligate precursor to IDC. Involvement of stromal cells, such as cancer-associated fibroblasts (CAFs), which secrete certain HGF and fibroblast activating protein (FAP) aid in DCIS cells become invasive and develop into IDC.

LCIS is a pre-cancerous neoplasia. It may be indicative of a predisposition for invasive cancer. LCIS only accounts for about 15% of the in situ (ductal or lobular) breast cancers. LCIS biomarkers include, but are not limited to, E-cadherin, ER, PgR, c-erbB-2, p53 and Ki-67.

IDC is the most invasive breast cancer. As the name applies, it is a carcinoma that begins in the breast ducts and then invades the surrounding fatty tissue. About 8 to 10 invasive breast cancers are infiltrating ductal carcinomas. IDC is often treated by surgery to excise the cancerous tissue, and radiation therapy. In addition, chemotherapy combined with immunotherapy (e.g., tamoxifen and tratuzumab) is often used to treat IDC. If the tumor is larger than 4 cm, then a radical mastectomy may be performed. Biomarkers for IDC, include but are not limited to, carbohydrate antigens such as Tn, Tf, sialyl-Tn, Lewis x, Lewis a, Lewis y, and gangliosides such as GM3, GD3, 9-0-acetyl GD3, 9-0-acetyl GT3, N-glycoly-GM3.

ILC is a cancer that develops in the lobules of the breast and has invaded the surrounding tissue. About 1 in 10 invasive breast cancer is an ILC. ILC is treated by surgery to excise the cancerous tissue, and radiation therapy. In addition, chemotherapy and immunotherapy combination (e.g., tamoxifen and tratuzumab) is often used as an adjuvant therapy to treat ILC. Like IDC which is invasive with the aid of growth factors and cytokines released by cancer-associated fibroblasts (CAFs), ILC too is aided by CAFs-related proteins FAP-α, FSP-1/S100A4, and PDGFR-β.

Inflammatory breast cancer accounts for about 1% to 3% of all breast cancers. In inflammatory breast cancer, cancer cells block lymph vessels in the skin resulting in the beast turning red and feeling warm. The affected breast may become larger or firmer, tender, or itchy. Inflammatory breast cancer is treated with chemotherapy, immunotherapy, radiation therapy and in some cases, surgery.

Estrogen Receptor positive (ER+) breast cancer is characterized by the presence of estrogen receptors on the surface of the cancerous cells. Growth of ER+ cancer cells is associated with the availability of estrogen (hormone-dependent or hormone sensitive breast cancer). Approximately, 80% of all breast cancers are ER+ breast cancers. Treatment options for ER+ breast cancer include chemotherapeutic agents that block estrogen (e.g., tamoxifen).

Triple negative breast cancer is characterized by the absence of estrogen receptor, progesterone receptor, and HER2 receptor and occurs in about 10%-20% of diagnosed breast cancers. TNBC can be very aggressive and a subject has a risk of reoccurrence. Treatment options typically includes neoadjuvant chemotherapy (but not an anti-estrogen such as tamoxifen or anti-HER2 such as trastuzumab) followed by surgery such as lumpectomy. Further, TNBC is a highly diverse group of cancers and has been subtyped into at least 6 TNBC subtypes displaying unique gene expression and ontologies, including 2 basal-like (BL1 and BL2), an immunomodulatory (IM), a mesenchymal (M), a mesenchymal stem-like (MSL), and a luminal androgen receptor (LAR) subtype (Lehrmann et al. J. Clin. Invest. 2011, 121(7), 2750-2767) incorporated by reference herein in its entirety. Mutations and markers have been described that are can target specifically the TNBC subtypes (Id.). Additional biomarkers for TNBC include, but are not limited to, Epidermal growth factor receptor, folate receptor-α, vascular endothelial growth factor, c-Myc, C-kit and basal cytokeratins, Poly(ADP-ribose) polymerase-1, p53, tyrosinase kinases, m-TOR, heat and shock proteins and TOP-2A. Further, stromal cells (cancer-associated fibroblasts (CAFs) and immune cells such as tumor associated macrophages (TAMs) and Tumor associated neutrophils (TANs)) surrounding tumors play an important role in creating barriers to therapeutic drug penetration by through increased deposition of extracellular matrix and release of various profibrotic growth factors such as TGF beta, bFGF, and other factors that impact cancer cell proliferation, invasion and metastasis (for example by promoting epithelial-to-mesenchymal transition) such as fibroblast activating protein (FAP), EGF, HGF, MCP-1, CSF-1, VEGF, cytokines such as IL1, IL-8, TNF-alpha, enzymes such as MM2, MMP7, MMP8, MMP9, MMP12, MMP13, and COX2. TAMs further suppress anti-tumor immune response by secreting cytokines and chemokines (for example IL-10, TGF-beta, CCL17, CCL22, and CCL24) favoring the recruitment of T-reg cells and generation of immune suppressive microenvironment.

In some embodiments, the subject's breast disorder is breast cancer or phyllodes tumor. In yet other embodiments, the breast cancer is a pre-cancer, an early stage cancer, a non-metastatic cancer, a pre-metastatic cancer, a locally advanced cancer, or a metastatic cancer.

In other embodiments, the subject has breast cancer selected from the group consisting of ductal carcinoma in situ (DCIS), lobular carcinoma in situ (LCIS), invasive (or infiltrating) lobular carcinoma (ILC), invasive (or infiltrating) ductal carcinoma (IDC), microinvasive breast carcinoma (MIC), inflammatory breast cancer, ER-positive (ER+) breast cancer, progesterone receptor positive (PR+) breast cancer, ER+/PR+ breast cancer, ER-negative (ER−) breast cancer, HER2+ breast cancer, triple negative breast cancer (i.e., ER−/PR−/Her2− breast cancer; “TNBC”), adenoid cystic (adenocystic) carcinoma, low-grade adenosquamatous carcinoma, medullary carcinoma, mucinous (or colloid) carcinoma, papillary carcinoma, tubular carcinoma, metaplastic carcinoma, and micropapillary carcinoma.

### Compositions

### Repolarizing Agent

In an aspect, the present disclosure provides compositions comprising repolarizing agents capable of repolarizing M2-macrophages. Without wishing to be bound by any theory or mechanism, the compositions comprising repolarizing agents repolarize or switch the TAMS from M2-macrophages activation phase to M1-macrophage activation phase.

A repolarizing agent can be any agent that promotes M1-macrophage phenotype. Examples of repolarizing agent include, without limitation, fenretinide (4-hydroxy(phenyl)retinamide, 4-HPR); IL-12; IFNγ; LPS, ferumoxytol, histidine-rich glycoprotein (HRG); miRNA-155, miRNA-125, miRNA-127, miRNA-17, miRNA-20, miRNA-106a, and miRNA-378;

inhibitors of colony stimulating factor-1 (CSF-1) and CSF-1 receptor (CSF-1R);

inhibitors of IL-10 (such as IL-12 and activators thereof), IL-10 receptor (IL-10R);

inhibitors of TGFβ (such as trabedersen [AP12009], Lucanix® [belagenpumatucel-L], disitertide, Lerdelimumab, Metelimumab, Fresolimumab, LY2382770 and the like (see also, Neuzillet et al. Pharmacology & Therapeutics, Vol. 147, March 2015, Pages 22-31 for additional examples of TGFβ inhibitors);

inhibitors of Arginase 1 (Arg1) such as CB-1158;

inhibitors of M2 macrophage scavenger receptors (such as A, B, MARCO (such as anti-MARCO antibodies, ScFvs and the like));

inhibitors of histone deacetylase (HDACi);

inhibitors of interferon regulatory factor-4 (IRF4), Signal transducer and activator of transcription 4 and 6 (STAT4; STAT6) signaling pathway such as fenretinide (4-HPR), leflunomid, TMX264, and AS1217499;

inhibitors of IL-4 (such as pitakinra, pascolizumab, dupilumab, formomonetin, budesonide, rocaglamide, mesopram, GIT-27) and IL-4R;

anti-IL-4/IL-13 inhibitors such as duplimab;

inhibitors of IL-13 (such as lebrikizumab, anrukinzumab, lebrikizunab and tralokinumab and IL-13R(alpha)2 decoy) and IL-13R;

inhibitors of IL-17 (such as secukinumab) and IL-17R (for example brodalumab, ixekizumab, bimekizumab, ALX-0761, CJM112, CNTO 6785, LY3074828, and SCH-900117);

inhibitors of peroxisome proliferator-activated receptor gamma (PPARγ) (such as rosiglitazone, pioglitazozne);

inhibitors of Krüppel-like factor 4 (KLF4) and Krüppel-like factor 6 (KLF6);

inhibitors of microRNA-processing enzyme DICER;

inhibitors of miRNA-9, miRNA-21 (such as AC1MMYR2), miRNA147, miRNA-187, miRNA-146 family members such as (miRNA-146a), let7 family members (such as let-7c);

inhibitors of CCR-CC12 axis signaling;

inhibitors of CCL2/MCP-1 synthesis;

inhibitors of placental growth factor (PlGF) and C/EBPβ (PI3Kγ deletion);

inhibitors of AMPKα1 (such as metformin);

inhibitors of p50-p50 NFκB (such as the decoy NFkB p50 (NLS) Inhibitor Peptide Set—Novus Biologicals);

inhibitors of NADPH oxidases 1 and 2 (NOX 1 and NOX 2) such as ML-171, GSK2795039, Bridged tetrahydroisoquinolines and the like;

inhibitors of Notch signaling pathway, for example inhibitors of RPB-J;

activators of CD40 such as activating antibodies for example CP-870,893, Dacetuzmumab, ChiLob7/4, vaccines, and the like) and CD40L (such as AdcuCD40L);

activators of interferon regulatory factor-1 (IRF1), interferon regulatory factor-5 (IRF5); Signal transducer and activator of transcription 1 (STAT1) such as IFNγ, vadimezan (DMXAA; Signal transducer and activator of transcription 3 (STAT3);

toll-like receptor (TLR) agonists (such as Imiquimod, synthetic unmethylated cytosine-guanine oligodeoxinucleotides (CpG-ODNs), TLR4 ligands); and

activators of p65-p50 NFκB and MyD88;

or any combination thereof.

In some embodiments, the compositions comprise one or more of HDACi as a suitable repolarizing agent. Examples of suitable HDACi include, without limitation, TMP195, MC1568, TMP269, an (aryloxopropenyl)pyrrolyl hydroxamate), trichostatin A, trapoxin B, tubastatin A hydrochloride (anti-HDAC7), Panobinostat, suberoylanilide hydroxamic acid (SAHA) a Class I and Class II inhibitor Vorinostat (Volinza® Merck), Romidepsin (Istodax®); Depsipeptide, FK-228), Belinostat (PXD-101), Panobinostat (LBH589), Dacinostat (LAQ824), SB939, Chidamide, pan-HDAC inhibitors (such as Givinostat (ITF2357), PCI 2478, R306465 (JNJ-16241199), Resminostat (4SC-201)), valproic acid, butyric acid, phenylbutyrate, AN9/Pivanex, Class I-selective HDAC inhibitors benzamides or amino analides (such as CI-994, Entinostat (SNDX-275/MS-275), Mocetinostat (MGCD0103), Abexinostat (PCI-24781), Quisinostat (JNJ-26481585), HBI-8000, Kevetrin, CUDC-101, AR-42, CHR-2845, CHR-3996, 4SC-202, CG200745, ACY-1215, ME-344, and Sulforaphane. In at least one embodiment, the suitable HDACi is TMP195.

MARCO is expressed by immunosuppressive TAMs in human breast cancer. Reprogramming or repolarization of M2-macrophages, specifically immunosuppressive TAMs is highly desirable. Accordingly, in some embodiments, the repolarizing gent is a MARCO inhibitor. MARCO inhibitor may be anti-MARCO antibodies (such as ab103311, monoclonal ED31, PLK-1, ABN 1389), anti-MARCO ScFv, Fab, Fab′, and Fab2, anti-MARCO ScFv mRNA, anti-MARCO miRNA, MARCO anti-sense RNA, MARCO siRNA, or any combination thereof. In at least one embodiment, the anti-MARCO inhibitor is an anti-MARCO antibody or an ScFv.

In an aspect, the present invention discloses that the administration intraductally of a composition comprising anti-MARCO antibody or ScFv to a breast duct of a subject is able to induce antibody-dependent cell death (ADCC) in M2-macrophage.

In at least one embodiment, the repolarization agent is IL-12.

### Blockading Agents

In an aspect, the present disclosure provides compositions comprising a polarization blockading agent capable of blocking the polarizing of infiltrating monocyte, breast tissue resident macrophages, M1-macrophages from becoming M2-polarized macrophages in the TME. Without wishing to be bound by any theory or mechanism, administration intraductally of the composition comprising a blockading agent reduces the number of M2-macrophages in the TME. In some embodiments, M1-macrophages are increased. In other embodiments, the ratio of M2-macrophages to M1-macrophages is altered. In still other embodiments, administration of the blockading agent results in increase in pro-inflammatory factors such as cytokines, chemokines, and growth factors and the like, such as IFNγ, IL-1, IL-1β, TNF-α, GM-CSF. In yet other embodiments, administration of the blockading agent results in decrease in anti-inflammatory factors such as cytokines, chemokines, and growth factors and the like, such as IL-4, IL-13, IL-10, TGFβ, VEGF, MMPs.

Further, blockading CSF-1 and CSF-1 receptor (CSF-1R) in breast cancer models has shown reduced monocyte and macrophage infiltration into the TME and increased cytotoxic CD8+ T cells in the tumors. This suggests that inhibiting CSF-1 and CSF-1R is correlated with increased CD8+ T cells in the tumors.

M2 polarization blockading agents suitable for the purpose of the present disclosure can be any polarization blockading agent that blocks the polarization of infiltrating monocyte, breast tissue resident macrophages, M1-macrophages from becoming M2-polarized macrophages. Examples include, without limitation, anti-CSF-1 inhibitors, anti-CSF-1R inhibitors, anti-MCP-1 inhibitors, anti-IL-4 inhibitors (such as pascolizumab, pitakinra and dupilumab), anti-IL-13 inhibitors (such as lebrikizumab, anrukinzumab, lebrikizunab and tralokinumab), anti-IL-4/IL-13 inhibitors such as duplimab, STAT3 inhibitors (such as sorafenib, sunitinib, WP1066, and resveratrol), and STAT6 inhibitors (such as fenretinide (4-HPR), leflunomid, TMX264, and AS1217499).

One of skill in the art will recognize that a repolarizing agent may be function as a polarization blockading agent.

Repolarizing agents suitable for the purpose of the present disclosure can be any type of repolarizing agent that repolarizes M2-macrophages to M1-macrophages. Likewise, polarization blockading agents suitable for the purpose of the present disclosure can be any type of repolarizing agent that block polarization of macrophages from becoming M2-macrophages. By way of non-limiting examples, such agents can be small molecule inhibitors, small molecule activators, genes, DNA, polynucleotides, oligonucleotides (ODNs), aptamers, dendrimers, copy DNA (cDNA), naked RNA, messenger RNA (mRNA), microRNA (miRNA), antisense RNA (ASR), silencer RNA (siRNA), long non-coding RNAs (lncRNA), proteins, polypeptides, peptides, peptidomimetics, decoy sequences, DNA vaccines, RNA vaccines, self-amplifying mRNA replicon, antibodies (human, humanized, chimeric, monoclonal, polyclonal, monospecific, bispecific, and the like), single chain variable fragment (ScFv), Fab fragment, Fab′, Fab2, saccharides, polysaccharides, gene therapy (such as CRISPR/Cas9-Mediated gene editing), and the like.

Proteins, polypeptides and peptides used to practice the invention can be isolated from natural sources, be synthetic, or be recombinantly generated polypeptides. Peptides and proteins can be recombinantly expressed in vitro or in vivo. The proteins, peptides and polypeptides used to practice the invention can be made and isolated using any method known in the art. Proteins, polypeptide and peptides used to practice the invention can also be synthesized, whole or in part, using chemical methods well known in the art. See e.g., Caruthers (1980) Nucleic Acids Res. Symp. Ser. 215-223; Horn (1980) Nucleic Acids Res. Symp. Ser. 225-232; Banga, A. K, Therapeutic Peptides and Proteins, Formulation, Processing and Delivery Systems (1995) Technomic Publishing Co., Lancaster, Pa. For example, peptide synthesis can be performed using various solid-phase techniques (see e.g., Roberge (1995) Science 269:202; Merrifield (1997) Methods Enzymol. 289:3-13) and automated synthesis may be achieved, e.g., using the ABI 431A Peptide Synthesizer (Perkin Elmer) in accordance with the instructions provided by the manufacturer.

Sequences for the repolarizing agents and polarization blockading agents (for example, Protein, polypeptide, gene, DNA, nucleotide sequences) can be determined as required from publicly and commercially available resources or known in the art such from NCBI Genbank and sequence viewer.

The polynucleotides may be delivered in various forms. In some embodiments, a naked polynucleotide may be used, either in linear form, or inserted into a plasmid, such as an expression plasmid. In other embodiments, a live vector such as a viral or bacterial vector may be used. One or more regulatory sequences that aid in transcription of DNA into RNA and/or translation of RNA into a polypeptide may be present. In some instances, such as in the case of a polynucleotide that is a messenger RNA (mRNA) molecule, regulatory sequences relating to the transcription process (e.g. a promoter) are not required, and protein expression may be effected in the absence of a promoter. One of skill in the art can include suitable regulatory sequences as the circumstances require.

In some embodiments, the polynucleotide is present in an expression cassette, in which it is operably linked to regulatory sequences that will permit the polynucleotide to be expressed in the subject to which the composition of the invention is administered. The choice of expression cassette depends on the subject to which the composition is administered as well as the features desired for the expressed polypeptide.

Typically, an expression cassette includes a promoter that is functional in the subject and can be constitutive or inducible; a ribosome binding site; a start codon (ATG) if necessary; the polynucleotide encoding the polypeptide of interest; a stop codon; and optionally a 3′ terminal region (translation and/or transcription terminator). Additional sequences such as a region encoding a signal peptide may be included. The polynucleotide encoding the polypeptide of interest may be homologous or heterologous to any of the other regulatory sequences in the expression cassette. Sequences to be expressed together with the polypeptide of interest, such as a signal peptide encoding region, are typically located adjacent to the polynucleotide encoding the protein to be expressed and placed in proper reading frame. The open reading frame constituted by the polynucleotide encoding the protein to be expressed solely or together with any other sequence to be expressed (e.g. the signal peptide), is placed under the control of the promoter so that transcription and translation occur in the subject to which the composition is administered.

In some embodiments, the repolarizing agent or the blockading agent comprises CRISPR-Cas9-assisted cassette used for in situ gene editing by CRISPR-Cas9-assisted cassette exchange in M2-macrophages.

Compositions will include a polarizing agent or a blocking agent or both cells in an amount effective to repolarize M2-macrophages or to block polarization of infiltrating monocytes and macrophages to M2-macrophage.

### Carriers

In another aspect, the present disclosure provides that compositions comprising a repolarizing agent or a blockading agent or both may further comprise a pharmaceutically acceptable carrier.

Carriers to be selected may be determined in part by the particular repolarizing agent or blockading agent and/or by the method of administration. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition.

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulations can include aqueous solutions.

In some embodiments, the carrier of the composition may comprise a continuous phase of a hydrophobic substance, such as a liquid hydrophobic substance. The continuous phase may be an essentially pure hydrophobic substance or a mixture of hydrophobic substances. In addition, the carrier may be an emulsion of water in a hydrophobic substance or an emulsion of water in a mixture of hydrophobic substances, provided the hydrophobic substance constitutes the continuous phase.

Hydrophobic substances that are useful in the compositions as described herein are those that are pharmaceutically acceptable. The carrier is preferably a liquid but certain hydrophobic substances that are not liquids at atmospheric temperature may be liquefied, for example by warming, and are also useful in this invention. In one embodiment, the hydrophobic carrier may be a Phosphate Buffered Saline.

Oil or water-in-oil emulsions are particularly suitable carriers for use in the present invention. Oils should be pharmaceutically acceptable. Suitable oils include, for example, mineral oils (especially light or low viscosity mineral oil such as Drakeol® 6VR), vegetable oils (e.g., soybean oil), nut oils (e.g., peanut oil), or mixtures thereof. In an embodiment, the oil is a mannide oleate in mineral oil solution, commercially available as Montanide® ISA 51. Animal fats and artificial hydrophobic polymeric materials, particularly those that are liquid at atmospheric temperature or that can be liquefied relatively easily, may also be used.

In embodiments herein where the composition is described as being a water-free liposome suspension (“water-free”), it is possible that the hydrophobic carrier of these “water-free” compositions may still contain small quantities of water, provided that the water is present in the non-continuous phase of the carrier. For example, individual components of the composition may have bound water that may not be completely removed by processes such as lyophilization or evaporation and certain hydrophobic carriers may contain small amounts of water dissolved therein. Generally, compositions of the invention that are described as “water-free” contain, for example, less than about 10%, 9%, 8%, 7%, 6%, 5%, 4%, 3%, 2%, 1%, 0.5%, 0.1%, 0.05% or 0.01% water on a weight/weight basis of the total weight of the carrier component of the composition.

In another aspect, the present disclosure provides that composition comprising a repolarizing or a blockading agent or both, are formulated as liposomes, microparticles, microspheres, nanocapsules, nanoparticles, nanospheres, lipid particles, vesicles, micelles, and exosomes. Accordingly, in some embodiments, composition comprising a repolarizing or a blockading agent or both, is comprised in liposomes, microparticles, microspheres, nanocapsules, nanoparticles, nanospheres, lipid particles, vesicles, micelles, and exosomes. In other embodiments, composition comprising a repolarizing or a blockading agent or both, is comprised on the surface of liposomes, microparticles, microspheres, nanocapsules, nanoparticles, nanospheres, lipid particles, vesicles, micelles, and exosomes. The exosomes may be harvested from subject's pro-inflammatory monocytes, peripheral blood derived monocytes and M1-macrophage as described by Tang et al. (FASEB J. 2016 September; 30(9):3097-3106).

Methods for making liposomes, nanoparticles, microparticles, microspheres, nanocapsules, nanospheres, lipid particles, vesicles, micelles, and exosomes are well known in the art.

Liposomes are completely closed lipid membranes containing an entrapped aqueous volume. Liposomes may be unilamellar vesicles (possessing a single bilayer membrane) or multilamellar vesicles characterized by multimembrane bilayers, each bilayer may or may not be separated from the next by an aqueous layer. As used herein and in the claims, the term “liposomes” is intended to encompass all such vesicular structures as described above, including, without limitation, those described in the art as “niosomes”, “transfersomes” and “virosomes.” The amphiphilic structure of liposome particles enables encapsulation of both hydrophilic and hydrophobic pharmaceutical drugs.

Methods for making liposomes are well known in the art. A general discussion of liposomes can be found in Akbarzadeh et al. Nanoscale Research Letters 2013, 8:102; Gregoriadis G. Immunol. Today, 11:89-97, 1990; and Frezard, F., Braz. J. Med. Bio. Res., 32:181-189, 1999. Any suitable method for making liposomes may be used in the practice of the invention, or liposomes may be obtained from a commercial source. Liposomes are typically prepared by hydrating the liposome components that will form the lipid bilayer (e.g. phospholipids and cholesterol) with an aqueous solution, which may be pure water or a solution of one or more components dissolved in water, e.g. phosphate-buffered saline (PBS), phosphate-free saline, or any other physiologically compatible aqueous solution.

Liposome compositions may be obtained, for example, by using natural lipids, synthetic lipids, sphingolipids, ether lipids, sterols, cardiolipin, cationic lipids and lipids modified with poly (ethylene glycol) and other polymers. Synthetic lipids may include the following fatty acid constituents; lauroyl, myristoyl, palmitoyl, stearoyl, arachidoyl, oleoyl, linoleoyl, erucoyl, or combinations of these fatty acids. Lipids particularly suitable for the making liposomes include, but are not limited to, phospholipids, dioleoylphosphatidylcholine (DOPC), dioleoyl phosphatidylethanolamine (DOPE), triolein, dipalmytoilphospatidylglycerol (DPPG), hydrogenated soy phospatidylcholine (HSPC), distearoylphospatidyl glycerol (DSPG), dioleoylphosphatidylcholine (DOPG), cholesterol, tricaprylin, triolein, N-(carbonyl-methoxypolyethylene glycol 2000)-1,2-distearoyl-sn-glycero-3-phospho-ethanolamine sodium salt (MPEG-DSPE), Lecithin, cephalin, sphingomyelin, egg phosphatidylcholine (EPC), disodium succinate hexahydrate (DSH), (1,2-bis(oleoyloxy)-3-(trimethylammonio) propane) (DOTAP), 1-[2-(oleoyloxy)ethyl]-2-oleyl-3-(2-hydroxyethyl)imidazolinium chloride (DOTIM), dimyristoylphosphatidylcholine (DMPC), and dimyristoylphosphatidylglycerol (DMPG).

Cationic synthetic lipids such as DOTAP and DOTIM are useful for the preparation of cationic liposomes having an affinity for tumor vasculature and breast ducts. In some embodiments, the making liposomes, nanoparticles, microparticles, microspheres, nanocapsules, nanospheres, lipid particles, vesicles, micelles are prepared using cationic synthetic lipids.

Although any liposomes may be used in this invention, including liposomes made from archaebacterial lipids, in at least one embodiment, phospholipids and unesterified cholesterol are used in the liposome formulation. The cholesterol is used to stabilize the liposomes and any other compound that stabilizes liposomes may replace the cholesterol. Other liposome stabilizing compounds are known to those skilled in the art. For example, saturated phospholipids produce liposomes with higher transition temperatures indicating increased stability.

Phospholipids that are preferably used in the preparation of liposomes are those with at least one head group selected from the group consisting of phosphoglycerol, phosphoethanolamine, phosphoserine, phosphocholine and phosphoinositol. In some embodiments, the liposomes comprise lipids which are 94-100% phosphatidylcholine. Such lipids are available commercially in the lecithin Phospholipon® 90 G. When unesterified cholesterol is also used in liposome formulation, the cholesterol is used in an amount equivalent to about 10% of the weight of phospholipid. If a compound other than cholesterol is used to stabilize the liposomes, one skilled in the art can readily determine the amount needed in the composition.

In other embodiments, a liposome component or mixture of liposome components, such as a phospholipid (e.g. Phospholipon® 90G), dioleoylphosphatidylcholine (DOPC), triolein, dipalmytoilphospatidylglycerol (DPPG), hydrogenated soy phospatidylcholine (HSPC), distearoylphospatidyl glycerol (DSPG), dioleoylphosphatidylcholine (DOPG), cholesterol, tricaprylin, triolein, N-(carbonyl-methoxypolyethylene glycol 2000)-1,2-distearoyl-sn-glycero-3-phospho-ethanolamine sodium salt (MPEG-DSPE), Lecithin, cephalin, sphingomyelin, egg phosphatidylcholine (EPC), disodium succinate hexahydrate (DSH), dimyristoylphosphatidylcholine (DMPC), dimyristoylphosphatidylglycerol (DMPG), may be solubilized in an organic solvent, such as a mixture of chloroform and methanol, followed by filtering (e.g. a PTFE 0.2 μm filter) and drying, e.g. by rotary evaporation, to remove the solvents.

Hydration of the resulting lipid mixture may be effected by, e.g., injecting the lipid mixture into an aqueous solution or sonicating the lipid mixture and an aqueous solution. During formation of liposomes, the liposome components form single bilaye.rs (unilamellar) or multiple bilayers (multilamellar) surrounding a volume of the aqueous solution with which the liposome components are hydrated.

In other embodiments, the liposomes may be combined with the carrier comprising a continuous hydrophobic phase.

If the carrier is composed solely of a hydrophobic substance or a mixture of hydrophobic substances (e.g. use of a 100% mineral oil carrier), the liposomes may simply be mixed with the hydrophobic substance, or if there are multiple hydrophobic substances, mixed with any one or a combination of them. If instead the carrier comprising a continuous phase of a hydrophobic substance contains a discontinuous aqueous phase, the carrier will typically take the form of an emulsion of the aqueous phase in the hydrophobic phase, such as a water-in-oil emulsion. Such compositions may contain an emulsifier to stabilize the emulsion and to promote an even distribution of the liposomes. Emulsifiers may be useful even if a water-free carrier is used, for the purpose of promoting an even distribution of the liposomes in the carrier. Typical emulsifiers include mannide oleate (Arlacel™ A), lecithin (e.g., S100 lecithin), a phospholipid, Tween™ 80, and Spans™ 20, 80, 83 and 85. In some embodiments, the volume ratio (v/v) of hydrophobic substance to emulsifier is in the range of about 5:1 to about 15:1. In at least one embodiment, the ratio is about 10:1.

The liposomes may be added to the finished emulsion, or they may be present in either the aqueous phase or the hydrophobic phase prior to emulsification. In some embodiments, the liposomes may be then dehydrated, such as by freeze-drying or lyophilization.

The repolarizing agent, the blockading agent, or both may be introduced at various different stages of the formulation process. More than one type of repolarizing or blockading agent may be incorporated into the composition (for example, an antibody and a small molecule inhibitor or a siRNA and an mRNA, a polypeptide and a RNA vaccine, etc.).

In some embodiments, the polarizing agent or the blockading agent or both are present in the aqueous solution used to hydrate the components that are used to form the lipid bilayers of the liposomes (e.g. phospholipid(s) and cholesterol). In this case, the polarizing agent or the blockading agent or both will be encapsulated in the liposome, present in its aqueous interior. If the resulting liposomes are not washed or dried, such that there is residual aqueous solution present that is ultimately mixed with the carrier comprising a continuous phase of a hydrophobic substance, it is possible that additional polarizing agent or the blockading agent may be present outside the liposomes in the final product.

In a related technique, the polarizing agent or the blockading agent or both may be mixed with the components used to form the lipid bilayers of the liposomes, prior to hydration with the aqueous solution. The polarizing agent or the blockading agent or both may also be added to pre-formed liposomes, in which case the antigen may be actively loaded into the liposomes, or bound to the surface of the liposomes or the antigen may remain external to the liposomes. In such embodiments, prior to the addition of polarizing agent or the blockading agent or both, the pre-formed liposomes may be empty liposomes (e.g. not containing encapsulated polarizing agent or the blockading agent) or the pre-formed liposomes may contain a polarizing agent or a blockading agent incorporated into or associated with the liposomes. These steps may occur before mixing with the carrier comprising a continuous phase of a hydrophobic substance.

In an alternative approach, the polarizing agent or the blockading agent or both may instead be mixed with the carrier comprising a continuous phase of a hydrophobic substance, before, during, or after the carrier is combined with the liposomes. If the carrier is an emulsion, the polarizing agent or the blockading agent or both may be mixed with either or both of the aqueous phase or hydrophobic phase prior to emulsification. Alternatively, the polarizing agent or the blockading agent or both may be mixed with the carrier after emulsification. In some embodiments, the polarizing agent or the blockading agent or both may be present within the liposomes and also in the carrier comprising a continuous phase of a hydrophobic substance.

If the compositions comprising polarizing agent or the blockading agent or both, further comprise additional therapeutic agents, then the additional therapeutic agents may be combined as described above.

Stabilizers such as sugars such as sucrose, anti-oxidants such as alpha-tocopherols, or preservatives that maintain the biological activity or improve chemical stability to prolong the shelf life of the repolarizing agents and blockading agents may be added to such compositions. Other excipients can include salts such as sodium chloride (NaCl) and calcium chloride, disodium phosphate dehydrate, potassium dihydrogen phosphate.

In an embodiment, to formulate a composition of the invention, a homogenous mixture of S100 lecithin and cholesterol (e.g., 10:1 w:w) are hydrated in the presence of a repolarizing agent or a blockading agent (such as anti-MARCO ScFV or anti-MARCO antibody, IL-12 mRNA, or TMP195) optionally in phosphate buffer, to form liposomes with encapsulated repolarizing agent or blockading agent. The liposome preparation may then be extruded, optionally through a manual mini-extruder, and optionally mixed with an additional therapeutic agent or an imaging agent. This suspension may then be lyophilized and reconstituted in a carrier comprising a continuous phase of a hydrophobic substance to form a water-free liposome suspension.

In some embodiments, the composition may be formulated by hydrating a homogenous mixture of at least one lipid component selected from the group consisting of triolein, dipalmytoilphospatidylglycerol (DPPG), hydrogenated soy phospatidylcholine (HSPC), distearoylphospatidyl glycerol (DSPG), dioleoylphosphatidylcholine (DOPG), cholesterol, tricaprylin, triolein, N-(carbonyl-methoxypolyethylene glycol 2000)-1,2-distearoyl-sn-glycero-3-phospho-ethanolamine sodium salt (MPEG-DSPE), Lecithin (0.01 mg/ml to 0.2 mg/ml), cephalin (0.005 mg/ml to 0.05 mg/ml), sphingomyelin, egg phosphatidylcholine (EPC), disodium succinate hexahydrate (DSH), dimyristoylphosphatidylcholine (DMPC), dimyristoylphosphatidylglycerol (DMPG) (e.g., 10:1 w:w) with a repolarizing agent, a blockading agent or both, optionally in phosphate buffer, to form liposomes encapsulated with a repolarizing agent, a blockading agent or both.

In other embodiments, the composition may be formulated by hydrating a homogenous mixture of at least two of the lipid components selected from the group consisting of triolein, dipalmytoilphospatidylglycerol (DPPG), hydrogenated soy phospatidylcholine (HSPC), distearoylphospatidyl glycerol (DSPG), dioleoylphosphatidylcholine (DOPG), cholesterol, tricaprylin, triolein, N-(carbonyl-methoxypolyethylene glycol 2000)-1,2-distearoyl-sn-glycero-3-phospho-ethanolamine sodium salt (MPEG-DSPE), Lecithin (0.01 mg/ml to 0.2 mg/ml), cephalin (0.005 mg/ml to 0.05 mg/ml), sphingomyelin, egg phosphatidylcholine (EPC), disodium succinate hexahydrate (DSH), dimyristoylphosphatidylcholine (DMPC), dimyristoylphosphatidylglycerol (DMPG) (e.g., 10:1 w:w) in the presence of a repolarizing agent, a blockading agent or both, optionally in phosphate buffer, to form liposomes encapsulated with a repolarizing agent, a blockading agent or both.

In some embodiments, the composition may be formulated by hydrating a homogenous mixture of dioleoyl-phosphatidylcholine (DOPC) and cholesterol (e.g., 10:1 w:w) in the presence of a repolarizing agent, a blockading agent or both, optionally in phosphate buffer, to form liposomes encapsulated with a repolarizing agent, a blockading agent or both.

In an aspect, the present disclosure provides that the compositions comprising a polarizing agent or a blockading agent or both is formulated as a nanoparticle. In some embodiments, such as nanoparticle may be a lipid nanoparticle (such as a modified liposome). Modified liposomes at the nanoscale have been shown to have excellent pharmacokinetic profiles for the delivery of DNA, antisense oligonucleotide, siRNA, proteins and chemotherapeutic agents. Accordingly, in some embodiments, the polarizing agents or blockading agents or both are comprised in lipid nanoparticles. In other embodiments, the polarizing agents or blocking agents or both are loaded on the surface of the nanoparticles (e.g., lipid particles such as lipid-coated nanoparticles, as shown in Example 1) or liposomes.

Other suitable nanoparticles include polymeric nanoparticles (for, e.g., fabricated using biodegradable synthetic polymers, such as polylactide-polyglycolide copolymers, polyacrylates and polycaprolactones, or natural polymers, such as albumin, gelatin, alginate, collagen and chitosan), polymeric micelles, hydrogel nanoparticles, dendrimers, noble metal nanoparticles (such as gold nanoparticles), and the like.

In some embodiments, the composition comprises nanoparticles with an average or mean diameter of no greater than about 1000 nanometers (nm), such as no greater than about any of 900, 800, 700, 600, 500, 400, 300, 200, and 100 nm. In some embodiments, the average or mean diameters of the nanoparticles is no greater than about 200 nm. In some embodiments, the average or mean diameters of the nanoparticles is no greater than about 150 nm. In some embodiments, the average or mean diameters of the nanoparticles is no greater than about 100 nm. In some embodiments, the average or mean diameter of the nanoparticles is about 20 to about 400 nm. In some embodiments, the average or mean diameter of the nanoparticles is about 40 to about 200 nm. In some embodiments, the nanoparticles are sterile-filterable.

In some embodiments, the release of repolarizing agents and blockading agents from nanoparticle- and liposome-responsive polymers, or hydrogel, may be triggered by a change in pH, temperature, radiofrequency or magnetic field.

In an aspect, the present disclosure provides that nanoparticles and liposomes are formulated for target-specific drug delivery. Such target specificity is effected by conjugating the nanoparticles and liposomes with one or more cell targeting agents that targets M2-macrophages. The cell targeting agents suitable for the purpose of this invention can be any M2-macrophage selective cell surface molecule. The present disclosure provides that nanoparticles and liposomes are coated with one or more of M2 macrophage selective cell surface molecule. One of skill in the art will recognize that M2-macrophage selective cell surface molecules may be present exclusively on M2-macrophages but may also be present on non-M-2 macrophages. However, suitable M2-macrophage selective cell surface molecules are those that are expressed exclusively on M2-macrophages or expressed at a higher level on M2-macriphages than on non-M2-macrophages. M2 macrophage selective cell surface molecule suitable for the purpose of this invention include, without limitation, IL-13Rα, CD163, CD206, CD200R, MerTK, scavenger receptor A, scavenger receptor B, MARCO, and F4/80.

The nanoparticles described herein may be present in a dry formulation (such as lyophilized composition) or suspended in a biocompatible medium. Suitable biocompatible media include, but are not limited to, water, buffered aqueous media, saline, buffered saline, optionally buffered solutions of amino acids, optionally buffered solutions of proteins, optionally buffered solutions of sugars, optionally buffered solutions of vitamins, optionally buffered solutions of synthetic polymers, lipid-containing emulsions, and the like.

Nanoparticles may be prepared by any of the methods known in the art (see Pal et al., Journal of Applied Pharmaceutical Science 01 (06); 2011: 228-234; Hu Y et al. Nanoparticles. Nano Letters. 2007; 7:3056-3064; Hu Y H et al. Biomacromolecules. 2009; 10:756-765; Lynn D M, Langer R. Journal of the American Chemical Society. 2000; 122:10761-10768).

Addition of polarizing agents and blockading agent may be effected by any suitable means known. By way of non-limiting examples, DNA sequences, linear or as plasmids and cassettes may be added to the liposomes, microparticles, microspheres, nanocapsules, nanoparticles, nanospheres, lipid particles, vesicles, micelles, and exosomes as described by Goncalves et al, Su et al.

The present disclosure provides that the lipid to nucleic acid ratio (LNR) of liposomes, microparticles, microspheres, nanocapsules, nanoparticles, nanospheres, lipid particles, vesicles, and micelles ranges from 30:1 to 2.5:1. In some embodiments, the LNR of liposomes, microparticles, microspheres, nanocapsules, nanoparticles, nanospheres, lipid particles, vesicles, and micelles ranges from 25:1 to 5:1. In at least one embodiment the LNR of liposomes, microparticles, microspheres, nanocapsules, nanoparticles, nanospheres, lipid particles, vesicles, and micelles ranges from 20:1 to 10:1.

The present disclosure provides that the lipid to protein ratio (LPR) of liposomes, microparticles, microspheres, nanocapsules, nanoparticles, nanospheres, lipid particles, vesicles, and micelles ranges from 30:1 to 1:1. In some embodiments, the LNR of liposomes, microparticles, microspheres, nanocapsules, nanoparticles, nanospheres, lipid particles, vesicles, and micelles ranges from 25:1 to 5:1. In at least one embodiment the LNR of liposomes, microparticles, microspheres, nanocapsules, nanoparticles, nanospheres, lipid particles, vesicles, and micelles ranges from 20:1 to 10:1.

The present disclosure provides that the small molecule drug to lipid (D/L) ratio of liposomes, microparticles, microspheres, nanocapsules, nanoparticles, nanospheres, lipid particles, vesicles, and micelles of the present invention typically is at least 0.05, 0.1, 0.2, 0.35, 0.5, or at least 0.65 mg of the drug per mg of lipid. In terms of molar ratio, the D/L ratio according to the present invention is at least from about 0.02, to about 5, from at least 0.1 to about 2, and from about 0.15 to about 1.5 moles of the drug per mole of the lipid.

In an aspect, the present disclosure provides that the compositions comprising a polarizing agent or a blockading agent, or both further comprises an imaging agent, a dye or a contrasting agent. Suitable imaging agents can be gadolinium chelates, superparamagnetic iron oxide nanoparticles (SPION), 19F perfluorocarbon nanoparticles, and other magnetic reporter genes, such as metalloprotein-based MRI probes. Addition of imaging agents, dyes and contrasting agents disclosed herein provide an added advantage of tracking the intraductal delivery of the compositions disclosed herein and migration of the macrophages that phagocytose these compositions.

In another aspect, the present disclosure provides that the compositions comprising a polarizing agent or a blockading agent, or both further comprises an additional therapeutic agent. Such additional therapeutic agent can be any agent useful for the treatment of a breast disorder. In some embodiments, the additional therapeutic agent is one or more of checkpoint inhibitors, anti-hormonals, steroids, anthracyclines, thyroid hormone replacement drugs, thymidylate-targeted drugs (such as docetaxel, gemcitabine, paclitaxel or carboplatin and pegylated liposomal doxorubicin), DNA hypomethylating agents (such as azacitidine or decitabine), trastuzumab, ado-trastuzumab emtansine, pertuzumab, abemaciclib, palbociclib, cell therapy such as Chimeric Antigen Receptor/T cell (CAR-T) therapies, and other adoptive cell therapies.

In some embodiments, the additional therapeutic agent is one or more of anti-hormonal selected from the group consisting of tamoxifen, cis-tamoxifen, endoxifen, desmethyltamoxifen, lasofoxifene, raloxifene, benzothiophene, bazedofoxifene, arzoxifene, miproxifene, levormeloxifene, droloxifene, clomifene, idoxifene, toremifene, EM652 and ERA-923, fulvestrant, ARN-810, or CH498, anastrozole, exemestane and letrozole.

In some embodiments, the additional therapeutic agent is one or more of checkpoint inhibitors include a checkpoint point inhibitor selected from the group consisting of anti-PD-1 (such as Nivolumab), anti-PD-1L (such as atezolizumab (MPDL3280), Avelumab (MSB0010718C), Durvalumab, MDX-1105), anti-CTLA4 (e.g., Ipilimumab), anti-LAG-3 (such as IMP321, BMS-986016 and GSK2831781), or a combination thereof.

In some embodiments, the compositions are formulated as depot formulations.

Compositions disclosed herein are suitable for intraductal administration to a breast duct of a subject.

### Mode of Delivery; Devices

In an aspect, the present disclosure provides that compositions comprising a repolarizing agent or a blockading agent or both may be delivered intraductally into one or more breast ducts of a subject by any of the methods known in the art. These include, but are not limited to, injections using syringe/needle (Krause et al. J. Vis. Exp. 2013; (80): 50692); cannula(e), catheters, probes, as well as those disclosed in U.S. Pat. Nos. 6,413,228; 6,689,070; 6,638,727, patent application PCT/US2015/010808), time-release capsules and encapsulation devices etc.

As a non-limiting example, in some embodiments, cells or compositions disclosed herein may be administered to a breast milk duct of the subject comprising (a) contacting a composition comprising modified cells, contained within a treatment chamber of a device with a nipple of a breast; and (b) applying positive pressure on the composition. In some embodiments, the composition is forced into the breast duct due to the positive pressure. Preferably, the composition is forced into one or more breast ducts. In other embodiments, the composition is forced into 2 to 5 breast ducts, into 4 to 8 breast ducts, or into 7 to 11 breast ducts.

For example, U.S. Pat. No. 6,413,228 discloses a ductal access device that is capable of collecting ductal fluid and infusing the ductal with wash fluid. Such a device can be adapted or configured appropriately for the purpose of this disclosure for the intraductal delivery of compositions of the present invention.

In some embodiments, the device is a cannula or a microcannula.

As an alternate method of intraductal administration, a small pump may be installed in the duct or at the surface of the nipple with access to the duct for slow continuous administration of modified cells to the ductal region e.g., a pump may be installed in the lactiferous sinus for administering the modified cells therein and causing a diffusion of the cells to the rest of the duct or the pump may be installed on the nipple surface with access to the duct. A pump installed at the nipple surface can be shaped, e.g., like a tack (or a thimble-shaped portion having a top or tack portion and the rest on the nipple surface with a portion extending into a duct requiring treatment or having a risk of requiring treatment. The pump mechanism can comprise, e.g., a Duros™ osmostic (micro)pump (Viadur), manufactured by Alza Corp acquired by Johnson & Johnson, IntelliDrug, Alzet® (Durect Corp.), Ivomec SR® bolus etc. (Herrlich et al. Advanced Drug Delivery Reviews. 2012, pages 1617-1626).

Osmotic pumps may also be assembled or configured essentially as the pumps described in U.S. Pat. Nos. 5,531,736, 5,279,608, 5,562,654, 5,827,538, 5,798,119, 5,795,591, 4,552,561, or 5,492,534, with appropriate modifications in size and volume for administration to the duct of a breast, e.g. for placement into the duct (e.g. the lactiferous sinus) or for placement on the nipple surface. The tip (that accesses the duct) may be able to rotate in order to accommodate ducts of various positions on the nipple surface. A single tack-head pump can have one or more tips placed below the tack-head in order to access a particular duct or ducts, e.g., where two or more ducts in a breast need to be accessed. The pump so configured and loaded with an appropriately formulated compositions comprising polarizing agent or a blockading agent or both for intraductal administration, may administer the compositions as described, but may also contain and administer agents other than the compositions disclosed herein for an appropriate therapeutic purpose for treatment of a precancer or cancer condition in a breast duct. Conceivably the pump may be configured to administer to all the ducts located in the breast, with some size and volume alterations.

Encapsulation devices are another attractive method and include microencapsulation and macroencapsulation devices. Self-folding immune-protective encapsulation devices have been developed wherein cells are immune-isolated by surrounding them with a synthetic semipermeable nanoporous membrane that allows selective permeation of nutrients and therapeutics. Such encapsulation devices include pouches, fibers, beads, and any device made from semipermeable materials within which the cells are housed. Such devices may be configured for therapy, for example, be prepared with biodegradable materials so as to release the cells within the duct.

Devices useful for the purpose of this invention may be implantable. For, e.g., Stephan et al. have described biopolymer implants for delivery of compositions (Stephan et al. Nature Biotechnology, 2015, 33, 97-101. In an aspect, provided herein are devices that are implantable, for example, cannula(e), microcannula(e), catheters, microcatheters, beads, encapsulation devices etc. Implantable devices for example may be configured to release cells and compositions of the present invention close to affected tissue and reduce their exposure to normal cells.

In another aspect, intraductal delivery of compositions disclosed herein may be aided with iontophoresis which involves application of an electric current to the breast which aid the migration of the cells into and/or within a duct of the breast.

### Dosing

The timing and size of dosing of compositions disclosed herein are generally designed to reduce risk of or minimize toxic outcomes and/or improve efficacy, such as providing faster and increased exposure of the subject to the compositions, e.g., over time. The quantity and frequency of administration will be determined by such factors as the condition of the patient, age, weight, tumor size and stage, surface area, and severity of the subject's disease, although the appropriate dosage may be determined by attending physician.

Optimal dosages and dosing regimen can be readily determined by a person of skill in the art of medicine by monitoring the patient for signs of disease and adjusting the treatment accordingly. Compositions can be administered multiple times at these dosages. Accordingly, the methods can involve a single dose or multiple doses over a period of time or continuous dose for e.g. by infusion. In some embodiments, a dose can be a single unit dose. In other embodiments, a single dose can be a split unit dose. As used herein, the term “split unit dose” refers to a unit dose that is split so that it is administered over more than one time during a day, including over more than one day. A split unit dose for the purpose of this invention is considered a single, i.e., one unit dose. Exemplary methods of splitting a dose include administering 25% of the dose the first day and administering the remaining the next day. In another embodiment, the unit dose may be split into 2, 50% each to be delivered on 2 consecutive days. In yet another embodiment, a split unit dose may be given on 2 alternate days. In still another embodiment, the unit dose may be split into 3 to be administered equally on 3 consecutive days.

In an aspect, the present invention provides that the compositions comprising any of liposomes, microparticles, microspheres, nanocapsules, nanoparticles, nanospheres, lipid particles, vesicles, and exosomes are administered intraductally at a concentration of 1×10⁴ to 1×10⁸ liposomes microparticles, microspheres, nanocapsules, nanoparticles, nanospheres, lipid particles, vesicles, or exosomes per unit dose.

Methods disclosed herein involve administering one or more consecutive doses of cells into a breast duct of a subject who may have received a first dose, and/or administering the first and one or more subsequent doses. The doses are administered in particular amounts and according to particular timing schedule and parameters.

In another aspect, the first dose is administered intraductally and any subsequent dose is administered by any suitable means, including intraductally, by injection and by infusion, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, intrathoracic, intracranial, or subcutaneous administration.

In some embodiments, the methods generally involve administering intraductally the first dose of compositions disclosed herein thereby reducing the disease burden. This may be followed by a subsequent dose of composition administered during a particular time of window with respect to the first dose or the administration of the subsequent dose to a subject having received a first dose. The first dose in some embodiments is relatively low. The amount of compositions administered and the timing of the doses of compositions are designed to improve one or more outcomes, such as reduction in the immunosuppression, reduction in tumor size and increase in pro-inflammatory cytokines, chemokines, M2-repolarization to M1-macropahges, recruitment of cytotoxic T-lymphocytes, the like.

In some embodiments, disclosed herein are methods involving administration of subsequent doses of composition at an increased number, and thus a higher dose, than the first/initial dose.

Where dosing regimen involves multiple doses, each dose may be administered daily, several times a day (twice, thrice, four times and the like), alternate days, every 2 days, 3 days, 5 days, 7 days, 14 days, 15 days, 28 days, monthly, quarterly, 6 monthly, and annually.

In some aspects, the timing of doses following initial dose is measured from the initiation of the initial (first) dose to the initiation of the next dose. In other embodiments, the timing of doses following initial dose is measured from the completion of the initial (first) dose.

The present invention encompasses the initial dose may be a split unit dose followed by a second dose administered thereafter. In some embodiments, a second or a subsequent dose may be a split unit dose. By way of a non-limiting example, a split unit dose may be administered over three days and the second unit dose is administered the very next day or it may be administered a year later. Initial dose is intended to create any limitations with regards to a subject in need of such a dose by imply that the subject has never before received a dose of cell therapy or even that the subject has not before received a dose of the same cells expressing the same recombinant receptor or targeting the same antigen.

Generally, compositions as described herein may be administered intraductally 1×10⁴ to 1×10⁸ of any of liposomes, microparticles, microspheres, nanocapsules, nanoparticles, nanospheres, lipid particles, vesicles, or exosomes per unit dose.

### Combination Therapy

The present invention contemplates that intraductal methods disclosed herein further comprise combination therapy. In an aspect, the intraductal method further comprises administering to the subject one or more additional therapeutic agent or therapy. The additional therapeutic agents may be administered to the subject by any suitable means known in the art, including without limitation, intraductally, topically, orally, nasally, parenterally by injection or infusion, subcutaneously, etc. The additional therapeutic agents may be comprised in the compositions disclosed herein or may be independently formulated. The order of administration of the therapeutic agents and/or therapy may be in any order of administration. For example, the compositions disclosed herein may be co-administered with an additional therapeutic agent or the additional therapeutic agent may be administered first or the additional therapeutic agent may be administered after compositions of the present invention are administered.

The additional therapeutic agents can be any that are useful for the purpose of this invention.

Exemplary additional therapeutic agents include, without limitation, checkpoint inhibitors, anti-hormonals, steroids, anthracyclines, thyroid hormone replacement drugs, thymidylate-targeted drugs (such as docetaxel, gemcitabine, paclitaxel or carboplatin and pegylated liposomal doxorubicin), DNA hypomethylating agents (such as azacitidine or decitabine), trastuzumab, ado-trastuzumab emtansine, pertuzumab, abemaciclib, palbociclib, cell therapy such as Chimeric Antigen Receptor/T cell (CAR-T) therapies, and other adoptive cell therapies.

Additional therapy may include surgery, radiation, chemotherapy, acupuncture, non-transpapillary adoptive cell therapy, etc. The methods disclosed herein may be used as a primary therapy, neoadjuvant therapy (for example, before surgery (such as mastectomy or lumpectomy) or chemotherapy), or adjuvant therapy (for illustrative purposes only, after chemotherapy or treatment with other methods of adoptive cell therapy).

### Articles of Manufacture

Also provided herein are articles of manufacture such as kits and devices, for the administration of the cells and compositions disclosed herein for adoptive cell therapy, and for storage and administration of the cells and compositions.

The articles of manufacture include a composition disclosed herein, one or more containers, packaging material, and a label or package insert generally including instructions for administration of the cells to a subject.

The containers contain one or more unit doses of the composition to be administered. In some embodiments, the article of manufacture comprises one or more containers, each containing a single unit dose of the compositions. The unit dose may be an amount or number of any of: liposomes, microparticles, microspheres, nanocapsules, nanoparticles, nanospheres, lipid particles, vesicles, and exosomes to be administered to the subject in the first dose or twice the number (or more) the nanoparticles, liposomes, and the like to be administered in the first or subsequent dose(s). It may be the lowest dose or lowest possible dose of the nanoparticles, liposomes and the like that would be administered to the subject in connection with the administration method.

In some embodiments, each of the containers individually comprises a unit dose of the composition that contains the same or substantially the same number of any of: liposomes, microparticles, microspheres, nanocapsules, nanoparticles, nanospheres, lipid particles, vesicles, and exosomes. Thus in some embodiments, each of the containers comprises the same or approximately or substantially the same number of any of: liposomes, microparticles, microspheres, nanocapsules, nanoparticles, nanospheres, lipid particles, vesicles, and exosomes. In some embodiments, the unit dose includes 1×10⁴ or more of any of: liposomes, microparticles, microspheres, nanocapsules, nanoparticles, nanospheres, lipid particles, vesicles, exosomes. In some embodiments, the unit dose includes 1×10⁴ to 1×10⁸ of any of: liposomes, microparticles, microspheres, nanocapsules, nanoparticles, nanospheres, lipid particles, vesicles, and exosomes.

In some embodiments, the articles of manufacture further include an additional therapeutic agent such as checkpoint inhibitors, anti-hormonals, steroids, anthracyclines, thyroid hormone replacement drugs, thymidylate-targeted drugs (such as docetaxel, gemcitabine, paclitaxel or carboplatin and pegylated liposomal doxorubicin), DNA hypomethylating agents (such as azacitidine or decitabine), trastuzumab, ado-trastuzumab emtansine, pertuzumab, abemaciclib, palbociclib, cell therapy such as Chimeric Antigen Receptor/T cell (CAR-T) therapies, and other adoptive cell therapies. Anti-hormonals can be selected from the group consisting of tamoxifen, cis-tamoxifen, endoxifen, desmethyltamoxifen, lasofoxifene, raloxifene, benzothiophene, bazedofoxifene, arzoxifene, miproxifene, levormeloxifene, droloxifene, clomifene, idoxifene, toremifene, EM652.

In some embodiments, the articles of manufacture further include an imaging agent, a dye or a contrasting agent selected from the groups consisting of gadolinium chelates, superparamagnetic iron oxide nanoparticles (SPION), 19F perfluorocarbon nanoparticles.

Suitable containers include, without limitation, for example, bottles, vials, syringes, and flexible bags, such as infusion bags. The containers may be formed from a variety of materials such as glass or plastic. In some embodiments, the container has one or more port, e.g., sterile access ports, for example, for connection of tubing or cannulation to one or more tubes, e.g., for transpapillary delivery and/or for connection for purposes of transfer to and from other containers, such as cell culture and/or storage bags or other containers.

The article of manufacture may further include a package insert or label with one or more pieces of identifying information and/or instructions for use. In some embodiments, the information or instructions indicates that the contents can or should be used to treat a breast disorder and/or providing instructions therefor. The label or package insert may indicate that the contents of the article of manufacture are to be used for treating the breast disorder. In some embodiments, the label or package insert provides instructions to treat a subject, e.g., the subject having a breast disorder, via a method involving the intraductal administration of a first and one or more subsequent doses of the cells, e.g., according to any of the embodiments of the provided methods. In some embodiments, the instructions specify administration, in a first dose, of one unit dose, e.g., the contents of a single individual container in the article of manufacture, followed by one or more subsequent doses at a specified time point or within a specified time window and/or after the detection of the presence or absence or amount or degree of one or more factors or outcomes in the subject.

As used herein, the terms “a,” “an,” and “the” include plural reference unless the context dictates otherwise.

As used herein, the term “about” refers to a measurable value such as an amount, a temporal duration and the like, is meant to encompass variations of ±20% or in some instances ±10%, or in some instances ±5%, in some instances ±1%, and in some instances ±0.1% from the specified value, as such variations are appropriate to perform the disclosed methods.

“Activation” as used herein refers to the state of a cell that has been sufficiently stimulated to induce detectable cellular proliferation. Activation can also be associated with induced cytokine production, and detectable effector functions. The term “activated T cells” refers to, among other things, T cells that are undergoing cell division.

As used herein, “adjuvant therapy” refers to a therapy that follows a primary therapy and that is administered to subjects at risk of relapsing. These are subjects who have a history of breast disorder and have been treated with another mode of therapy. Adjuvant systemic therapy in case of breast cancer usually begins soon after primary therapy to delay recurrence, prolong survival or cure a subject. As used herein “primary therapy” refers to a first line of treatment upon initial diagnosis of a breast disorder in a subject. Non-limiting exemplary primary therapies may involve surgery, a wide range of chemotherapies, and radiotherapy.

As used herein, the terms “subject,” “patient,” and “individual,” may be used interchangeably herein and refer to a mammal such as a human. Mammals also include pet animals such as dogs, cats, laboratory animals, such as rats, mice, and farm animals such as cows and horses. Unless otherwise specified, a mammal may be of any gender or sex.

As used herein, a “route of administration” or “route of delivery” compositions of present disclosure refers to the pathway for delivering the compositions of the present disclosure to a subject.

### EXAMPLES

### Example 1

### Synthesis of Lipid-Coated Nanoparticles

Preparation of Lipid-coated Nanoparticles. Lipid-coated nanoparticles with a poly-1 core will be synthesized as described by Su et al. (Molecular Pharmaceutics 8, no. 3 (Jun. 6, 2011):774-787). Briefly, Lipid-coated nanoparticles with a poly-1 core will be synthesized via two different processes: a double emulsion/solvent evaporation approach or a solvent diffusion/nanoprecipitation strategy. For double emulsion synthesis, 30 mg of poly-1 (or PLGA for pH-insensitive control particles) and 2 mg of the phospholipids DOPC, DOTAP, and DSPE-PEG in a 7:2:1 molar ratio will be co-dissolved in 1 ml of dicholoromethane (DCM). 200 μl Phosphate buffered saline (PBS) will be then added to the mixture on ice during a 1 min sonication step at 7 W using a probe tip sonicator (Misonix XL2000, Farmingdale, N.Y.) to form a first emulsion. The primary emulsion will be then dispersed into 6 ml of distilled, deionized nuclease-free water with sonication at 12 W for 5 min, before leaving on a shaker for 18 h at 25° C. to evaporate the organic solvent. For nanoprecipitation synthesis, the same quantities of DOPC, DOTAP, and polymer will be co-dissolved in 4 ml of ethanol and added drop-wise to 40 ml of distilled, deionized nuclease-free water, followed by gentle stirring for 5 h to evaporate ethanol. DSPE will be introduced into the lipid coating via a post-insertion process: DSPE lipid will be added at 1 mM to 0.5 mg/ml particles in distilled, deionized nuclease-free water and the suspension will be stirred for 16 h at 25° C. The particles will be collected and washed once via centrifugation, resuspended in fresh water and stored at 4° C. until use. Lipid-free PVA stabilized particles will be prepared by similar processes except the organic emulsion or solution containing polymer only will be dispersed into a 2% w/vol PVA aqueous solution.

A fraction of each particle batch will be dried in a vacuum oven to determine the particle concentration (mg/ml) by measuring the dry mass. Dynamic light scattering (DLS) and zeta potential measurements will be used to determine the particle size and surface charge using a ZetaPALS dynamic light scattering detector (Brookhaven Instruments). To estimate the percentage of lipid incorporated and the resultant mol % of lipid present in the lipid surface coating, lipid-enveloped particles will be prepared by nanoprecipitation as described above with 1 mol % DOPE-rhodamine added as a tracer for measuring the total amount of lipid incorporated into the particles, before the post-insertion of a DSPE-lipid labeled with carboxyfluorescein (DSPE-CF). The lipids will be then stripped from the particle surface by treatment with 2% triton X-100 for 15 min and the supernatant will be measured for both rhodamine and fluorescein signals to quantitate the amount of lipid incorporated.

To investigate the surface structure of lipid-coated nanoparticles by cryoelectron microscopy (cryoEM), particles will be embedded in ice by blotting a particle suspension (3 uL) on a 1.2/1.3 μm holey carbon-coated copper grid (Electron Microscopy Sciences) and immediately freezing the sample in liquid ethane using a Leica plunge-freezing machine. Samples will be transferred to a cryogenic holder and imaged using a JEOL 2200FS transmission electron microscope at 185 μA emission current and 40 000× magnification.

Preparation of IL-12 mRNA Nanoparticles. Synthetic IL-12 mRNA will be prepared using the method described by Kavanaugh et al. (Blood. 2006; 107:1963-1969). Briefly, linearized plasmid DNA bearing the T7 promoter, an open reading frame (GFP, 811 nucleotides or firefly luciferase, 1714 nucleotides), and poly-A tail will be used as a template for in vitro transcription using the Message Machine T7 Ultra Transcription kit (Ambion).

Alternatively, enhanced GFP (EGFP) will be replaced by IL-12 gene in the pGEM4Z/EGFP/A64 vector, which will be used for in vitro mRNA transcription (IVT). The IL-12elasti cassette containing the p35 and p40 subunits of IL-12 joined together by a flexible linker (InvivoGen, San Diego, Calif., USA) will be used. This construct provides equal expression of both subunits and prevents over-expression of p40 and the creation of p40 homodimers which behave as an antagonist of IL-12p70.

The mRNA product will be precipitated with LiCl, resuspended in nuclease-free water, and quantitated with a NanoDrop spectrophotometer. RNA size, purity and integrity will be ascertained with an Agilient Bioanalyzer 2100. An RMA cell line electroporated with these mRNA transcripts will also be used to confirm the functionality of these mRNA in vitro. mRNA will be labeled with a fluorescent Cy3 tag using a Label IT® nucleic acid labeling kit (Minis Bio LLC) according to the manufacturer's protocol.

RNA Loading and Release. IL-12 mRNA will be loaded onto the surface of lipid-coated nanoparticles by first diluting the particle suspension to 1.5 mg/ml in nuclease-free water, then adding 200 pi of the diluted suspension dropwise to 100 μl containing 4 μg of RNA under gentle vortexing. The vial will be then incubated at 4° C. for 2 h on a rotator to allow RNA adsorption. Bound RNA will be determined indirectly by measuring fluorescence of either Cy3-labeled or ribogreen-stained RNA remaining in the supernatant after centrifugation of the particles using a fluorescence plate reader (SPECTRAmax, Molecular Devices Corp.). RNA bound to the particles will be determined by subtracting the quantity of RNA detected in the supernatant from the quantity measured in identically-treated control vials containing RNA solutions but no particles. To assess the binding kinetics and capacity of the particles, the particle concentration will be fixed at a suitable concentration (e.g., 1 mg/ml) while the binding time and RNA amount will be varied. Binding efficiency will be defined as the percentage of RNA initially present in solution that bound to the particles. Loading will be defined as the amount of RNA bound (μg) per mg of particles.

Release kinetics of bound RNA will be determined as described by Su et al (Molecular Pharmaceutics 8, no. 3 (Jun. 6, 2011): 774-787). To determine the release kinetics of bound RNA from nanoparticles in RPMI 1640 culture medium containing 10% FBS, aliquots of particle-adsorbed poly I:C (70 μg particles containing 1.87 μg poly I:C) will be resuspended in 140 μl of serum containing-media and incubated at 37° C. under gentle mixing. At each designated timepoint, an aliquot will be removed and particles will be washed once with nuclease free water before resuspending in a digestion buffer (100 mM sodium acetate, 2% triton X-100 and 1 mg/ml Poly (L-aspartic acid)) to dissolve the particles and disrupt any lipid-RNA or poly-1-RNA complexes. The amount of RNA remaining on the particles will be then determined by measuring the fluorescence following staining with ribogreen as above, and the amount of RNA released will be calculated by subtracting the amount of remaining RNA from the original amount.

### Example 2

### Intraductal Administration of Repolarizing Agent IL-12

Nipple Preparation. After subjects have disrobed, a clinician will clean the nipple on the breast to be studied. This includes wiping the nipple clean with a slightly granular gel or ointment to loosen and remove any dead skin cells and accumulated oils. This is a cleanser frequently used in hospitals before medical procedures. Afterwards, some numbing cream will be applied to the nipple.

Nipple Anesthetic. 1 mL of Lidocaine mixed with 0.1-0.2 mL of blue dye will be injected with a very small needle into the base of the nipple.

Duct Identification. After dye injection and before the catheter placement, a small, flexible wire will be inserted about ½ inch into the opening to further identify and dilate the duct opening. Once a duct is identified, a small piece of knotted suture material will be inserted into the duct to mark it. This will be done on at least 3 duct openings and as many as 5. If the clinician is unable to find at least 3 duct openings, the subject will be not able to continue in the study and will be withdrawn. These subjects will be replaced by newly enrolled subjects.

Catheter Placement, Instillation of nanoparticles (IL-12), and X-ray Examination. Once all of the nipple duct openings are marked, the clinician will insert and place a catheter via the ductal orifice into each marked breast duct so marked. Once the catheters are in place, the clinician may optionally slowly (over 30 seconds) instills less than 1 mL of radio-opaque dye into the ducts to permit imaging of the ducts.

After dye instillation and depending on the number of ducts identified, up to 2 mL (generally ranging from 0.5 to 2 mL) of a suspension of IL-12 nanoparticles (1×106) will be slowly (over 1 minute) injected into each duct. Only the breast containing the invasive cancer will be treated. Cells are intraductally administered into affected breast ducts in volumes upto 10 mL in batches or sets depending on the number of ducts to be treated. Attempts will be made to distribute the doses evenly duct-by-duct based upon the number of affected ducts or lobules identified.

The catheter generally will remain in each duct for approximately 1-5 minutes. During the procedure, subjects are asked to assess their pain using a visual analog scale. After dye and nanoparticles have been instilled an image will be taken to demonstrate adequate infusion of modified cells into the ducts. When the number of ducts affected is higher than 2 ducts, the intraductal therapy procedure may be in done in sets. The catheters will be removed from the first set of ducts, for example 2 adjacent duct, and these ducts will be each marked with a small piece of knotted suture material. At this point, subjects will be assessed for pain using the pain scale for pain assessment.

Subsequently, new catheters will be inserted into the remaining marked ducts. After the next half of the nipple ducts have been cannulated, and dye and cells will be infused, another image will be taken with the fluoroscope to document the ducts. Subjects will be asked again to assess their pain. The catheters will be removed and ducts individually marked with a small piece of knotted suture material. Benzoin ointment and a clear plastic dressing (bio-occlusive) will be placed on the nipple to keep the markers in place until surgery. The total procedure takes 0.5 to 1.5 hours. Photographs will be taken of the procedure.

If during assessment of pain, the subject reports Grade 3 or 4 pain in the breast which does not resolve within 10 minutes after infusion of the nanoparticles comprising IL-12, study related procedures will be discontinued for that subject. Blood draws and follow-up assessment as well as pathological assessment as described herein will be performed per the protocol. In this case, subjects are replaced in the study group for statistical purposes.

If no initial X-ray examination perforation is noted side effects will be assessed immediately. If the subject does not report any untoward effects, the remaining ducts will be cannulated and administered with nanoparticles. Study related blood draws and assessment as well as pathological assessment as described below will be performed per protocol.

In vitro Assays—Cytokine release. Subject's serum cytokines levels will be measure to determine changes in the inflammatory status of the TAMs in the subject. Serum cytokine levels of IL1A, IL1B, IL2, IL4, IL6, IL8, IL10, IL12, IL17A, IFNγ, TNFα and GM-CSF will be assayed before the intraductal delivery of the nanoparticles at day 0, and after intraductal administration of the nanoparticles at 4 hours, 8 hours, 12 hours, 24 hours, 36 hours, 48 hours, and 72 hours using commercially available Human Inflammatory Cytokines Multi-Analyte ELISArray Kit MEH-004A from Qiagen, Germantown, MD. Changes in IL-13 secretion will be determined using a commercially available IL-13 secretion ELISA assay such as those form Miltenyi Biotec and Thermo Fischer or Human IL-13 Luminex Performance Assay from R&D Biosystems. Cytokine secretion will be measured in samples diluted to be in the linear range of the assay. Time-dependent increasing levels of proinflammatory cytokines and chemokines will be indicative of increase of M1-like pro-inflammatory macrophages in the subject. Time-dependent decrease in the levels of IL-4, IL-13 and IL-10 will be indicative of decrease in number and/or activity M2-macrophages.

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable, and/or equivalent structures, functions, ranges, or steps to those claimed, whether or not such alternate, interchangeable, and/or equivalent structures, functions, ranges, or steps are disclosed herein, and without intending to publicly dedicate any patentable subject matter.

It is to be understood that compositions comprising endoxifen free base and salts thereof disclosed herein may be prepared with synthetically prepared endoxifen as well as isolated endoxifen. It is to be further understood that dosing of the subjects is based on amount of (Z)-endoxifen present in the composition.

All publications, patents and patent applications referred to herein are incorporated by reference in their entirety to the same extent as if each individual publication, patent or patent application was specifically and individually indicated to be incorporated by reference in its entirety.

The embodiments of the invention in which an exclusive property or privilege is claimed are defined as follows:

1. A method of treating a subject having a breast disorder, the method comprising delivering intraductally to the subject a composition comprising a repolarizing agent capable of repolarizing a M2-polarized macrophage, or a blockading agent capable of blocking M2 polarization of macrophages, or both.

2. A method of promoting increased sensitivity to chemotherapy in a subject having a breast disorder, comprising: administering intraductally to the subject a composition comprising a repolarizing agent or a blockading agent or both, wherein administration of the composition promotes increased sensitivity to chemotherapy.

3. A method for selective reduction of M2 macrophages in a subject having a breast disorder comprising intraductally administering to the subject a composition comprising a repolarizing agent or a blockading agent or both.

4. The method of any of the preceding claims, wherein the M2 macrophage phenotype is any one or more of M2a phenotype, M2b phenotype, and M2c phenotype, or a combination thereof.

5. The method of any of the preceding claims, wherein the intraductal administration of the composition comprising a repolarizing agent or a blockading agent or both results in one or more of:
a. decreased M2 macrophages;
b. increased M1-macrophages;
c. M1/M2-macrophage homeostasis;
d. decreased release of anti-inflammatory cytokines, chemokines or growth factors;
e. increased release of pro-inflammatory cytokines, chemokines, or growth factors;
f. increased tumoricidal activity of macrophages;
g. increased cytotoxic T-lymphocyte infiltration in to TME; and
h. increased T-cell activation.

6. The method of any of the preceding claims, wherein the intraductal administration of a composition comprising a repolarizing agent or a blockading agent or both results in a decrease in any one or more of a macrophage population selected from the group consisting of:
a. F4/80+ macrophages;
b. Grl− macrophages;
c. CD63+ macrophages
d. CD206+ macrophages;
e. CD200R cells/macrophages;
f. MARCO+ macrophages;
g. CD68+/CD68+ macrophages;
h. CD68+/CD163+ macrophages;
i. Tie2R+ cells/macrophages;
j. CD11b+/VEGF-R1+ macrophages;
k. CCR2+ myeloid cells/macrophages;
l. MerTK+ macrophages;
m. CD11b^{low}/MHCII^{high}/CCR2+/F4/80+/CD64+/MerTK+ macrophages;
n. CD11b+Grl−/F4/80+ macrophages;
o. CD45+/CD11b+/Ly6G−/Ly6C^{low}/F4/80+ macrophages;
p. CD11b+/F4/80+/MHCII+/Ly6C− macrophages;
q. CD45+/CD11b+/F4/80+/Tie2+/CD31− macrophages; and
r. CD45+/F480+/Tie2−/CD31− macrophages.

7. The method of any of the preceding claims, wherein the intraductal administration of a composition comprising a repolarizing agent or a blockading agent or both results in an increase in any one or more of a macrophage population selected from the group consisting of:
a. CD11b^{high}/MHCII^{high} macrophages;
b. HLA-DRα+ macrophages;
c. CD64+ macrophages;
d. CD86+ macrophages;
e. CD80+ macrophages;
f. CD68+/CD80+ macrophages;
g. CD64+ macrophages; and
h. Ly6C^{high}/CX3CR1^{high}/CCR2−/CD62L−/CD43^{low}(Ly6C^{high}) macrophages.

8. The method according to any of the preceding claims, wherein the intraductal administration of a repolarizing agent or a blockading agent or both, reduces in the subject any one or more of
a. tumor angiogenesis;
b. tumor invasion;
c. metastasis;
d. immunosuppression;
e. chemoresistance; and
f. release of anti-inflammatory cytokines, chemokines and growth factors.

9. The composition of any of the preceding claims, wherein the repolarizing agent is selected from the group consisting of fenretinide (4-hydroxy(phenyl)retinamide, 4-HPR); IL-12; IFNγ, miR127, miR155, and miR223, ferumoxytol, inhibitors of: CSF-1, CSF-1R, IL-10, IL-10R, TGFβ, Arginase 1 (Arg1), M2 macrophage scavenger receptors (such as A, B, MARCO); histone deacetylase (HDACi), DICER, IRF4/STAT4/STAT6 signaling pathway; IL-4, IL-13, IL-17, PPARγ, KLF4, KLF6; miRNA-146 family members such as (miRNA-146a), let7 family members (such as let-7c), miRNA-9, miRNA-21, miRNA-47, miRNA-187; CCR-CC12 axis signaling; CCL2/MCP-1 synthesis; placental growth factor (PlGF) (HRG) and C/EBPβ (PI3Kγ deletion); AMPKα1 (metformin), p50-p50 NFκB, NADPH oxidase (NOX) (NOX 1 and NOX 2), Rbpj, Notch signaling pathway; activators of CD40 and CD40L; IRF1, IRF5, STAT1 (such as IFNγ, vadimezan (DMXAA)) and STAT3; nuclear factor kappa B activators, toll-like receptor (TLR) agonists such as Imiquimod, synthetic unmethylated cytosine-guanine (CpG) oligodeoxinucleotides (CpG-ODNs), p65-p50 NFκB, MyD88, miR127, miR155, and miR223, or a combination thereof.

10. The composition of claim 9, wherein one or more HDACi is selected from the group consisting of TMP195, MC1568, TMP269, an (aryloxopropenyl)pyrrolyl hydroxamate), trichostatin A, trapoxin B, tubastatin A hydrochloride (anti-HDAC7), Panobinostat, suberoylanilide hydroxamic acid (SAHA) a Class I and Class II inhibitor Vorinostat (Volinza® Merck), Romidepsin (Istodax®); Depsipeptide, FK-228), Belinostat (PXD-101), Panobinostat (LBH589), Dacinostat (LAQ824), SB939, Chidamide, pan-HDAC inhibitors (such as Givinostat (ITF2357), PCI 2478, R306465 (JNJ-16241199), Resminostat (4SC-201)), valproic acid, butyric acid, phenylbutyrate, AN9/Pivanex, Class I-selective HDAC inhibitors benzamides or amino analides (such as CI-994, Entinostat (SNDX-275/MS-275), Mocetinostat (MGCD0103), Abexinostat (PCI-24781), Quisinostat (JNJ-26481585), HBI-8000, Kevetrin, CUDC-101, AR-42, CHR-2845, CHR-3996, 4SC-202, CG200745, ACY-1215, ME-344, and Sulforaphane, or a combination thereof.

11. The composition of claim 9, wherein the MARCO inhibitor is selected from the group consisting of anti-MARCO antibodies (such as ab103311, monoclonal ED31, PLK-1, ABN 1389), anti-MARCO ScFv, Fab, Fab′, and Fab2, anti-MARCO ScFv mRNA, anti-MARCO miRNA, MARCO antisense RNA, MARCO siRNA, anti-MARCO DNA, anti-MARCO oligonucleotides, anti-MARCO peptide inhibitors, or a combination thereof.

12. The composition of any of preceding claims, wherein one or more blockading agent is selected from the group consisting of anti-CSF-1 inhibitors, anti-CSF-1R inhibitors, anti-MCP-1 inhibitors, anti-IL-4 inhibitors (such as pascolizumab, pitakinra and dupilumab), anti-IL-13 inhibitors (such as anrukinzumab, lebrikizunab and tralokinumab), anti-IL-4/IL-13 dual inhibitors such as duplimab, STAT3 inhibitors (such as sorafenib, sunitinib, WP1066, and resveratrol), and STAT6 inhibitors (such as fenretinide (4-HPR), leflunomid, TMX264, and AS1217499), or a combination thereof.

13. The composition of any of the preceding claims, wherein the composition further comprises a pharmaceutically acceptable carrier.

14. The composition of any of the preceding claims, wherein the composition further comprises an additional therapeutic agent.

15. The composition of any of the preceding claims, wherein the additional therapeutic agent is selected from the group consisting of checkpoint inhibitors, anti-hormonals, steroids, anthracyclines, thyroid hormone replacement drugs, thymidylate-targeted drugs (such as docetaxel, gemcitabine, paclitaxel or carboplatin and PEGylated liposomal doxorubicin), DNA hypomethylating agents (such as azacitidine or decitabine), trastuzumab, ado-trastuzumab emtansine, pertuzumab, abemaciclib, palbociclib, cell therapy such as Chimeric Antigen Receptor/T cell (CAR-T) therapies, and other adoptive cell therapies

16. The composition of any of the preceding claims, wherein the anti-hormonal is selected from the group consisting of tamoxifen, cis-tamoxifen, endoxifen, desmethyltamoxifen, lasofoxifene, raloxifene, benzothiophene, bazedofoxifene, arzoxifene, miproxifene, levormeloxifene, droloxifene, clomifene, idoxifene, toremifene, EM652 and ERA-923, fulvestrant, ARN-810, or CH498, anastrozole, exemestane and letrozole, or a combination thereof.

17. The composition of any of the preceding claims, further comprising a checkpoint point inhibitor selected from the group consisting of anti-PD-1 (such as Nivolumab), anti-PD-1L (such as atezolizumab (MPDL3280), Avelumab (MSB0010718C), Durvalumab, MDX-1105), anti-CTLA4 (e.g., Ipilimumab), and anti-LAG-3 (such as IMP321, BMS-986016 and GSK2831781), or a combination thereof.

18. The composition of any of the preceding claims, wherein the composition further comprises an imaging agent, a dye or a contrasting agent selected from the groups consisting of gadolinium chelates, superparamagnetic iron oxide nanoparticles (SPION), ¹⁹F perfluorocarbon nanoparticles, and other magnetic reporter genes, such as metalloprotein-based MRI probes.

19. The composition of any of the preceding claims, wherein the composition is formulated as a liposome, a nanoparticle, a microparticle, a microsphere, a nanocapsule, a nanosphere, a lipid particle, a vesicle, a micelle, or an exosome.

20. The composition of claim 19, wherein the polarizing agent or the blockading agent or both are comprised in a liposome, a microparticle, a microsphere, a nanocapsule, a nanoparticle, a nanosphere, a lipid particle, a vesicle, a micelle, or an exosome.

21. The composition of claim 19 or claim 20, wherein the polarizing agent or the blockading agent or both are comprised on a liposome, a microparticle, a microsphere, a nanocapsule, a nanoparticle, a nanosphere, a lipid particle, a vesicle, a micelle, or exosomes.

22. The composition of any of claims 19 to 21, wherein the nanoparticle is a lipid nanoparticle.

23. The composition of any of claims 19 to 22, wherein the nanoparticle is further coated with a cell targeting agent.

24. The composition of claim 23, wherein the cell targeting agent targets a M2-macrophage selective cell surface molecule.

25. The composition of claim 24, wherein the M2-macrophage specific cell surface molecule is selected from the group consisting of IL-13Rα, CD163, CD206, CD200R, MerTK, scavenger receptor A, scavenger receptor B, MARCO, and F4/80.

26. The composition of any of the preceding claims, wherein the composition is formulated as a depot formulation.

27. The method of any of the claims 19 to 26, wherein subject is administered intraductally a composition comprising 1×104 to 1×108 liposomes, microparticles, microspheres, nanocapsules, nanoparticles, nanospheres, lipid particles, vesicles, or exosomes per unit dose.

28. The method according to any of the preceding claims, wherein the composition comprising the repolarizing agent or the blockading agent or both are administered in a single dose or multiple doses.

29. The method of any of the preceding claims, wherein the breast disorder is a breast cancer.

30. The method of any of the preceding claims, wherein the breast cancer is selected from the group consisting of ductal carcinoma in situ (DCIS), lobular carcinoma in situ (LCIS), invasive (or infiltrating) lobular carcinoma (ILC), invasive (or infiltrating) ductal carcinoma (IDC), microinvasive breast carcinoma (MIC), inflammatory breast cancer, ER-positive (ER+) breast cancer, progesterone receptor positive (PR+) breast cancer, ER+/PR+ breast cancer, ER-negative (ER−) breast cancer, HER2+ breast cancer, triple negative breast cancer (i.e., ER−/PR−/Her2− breast cancer; “TNBC”), adenoid cystic (adenocystic) carcinoma, low-grade adenosquamatous carcinoma, medullary carcinoma, mucinous (or colloid) carcinoma, papillary carcinoma, tubular carcinoma, metaplastic carcinoma, or micropapillary carcinoma.

31. The method of any of the preceding claims, further comprises administering a chemotherapy, a radiotherapy, or a cell therapy to the subject.

32. The method of claim 31, wherein the chemotherapy, radiotherapy, or cell therapy, or a combination thereof reduces tumor size or immunosuppression or both.

33. An article of manufacture, comprising a composition comprising a repolarizing agent or a blockading agent or both, one or more containers, packaging material, a label or package insert, and optionally, a device.

34. The article of manufacture of claim 33, wherein the device is a needle and syringe, a cannula, a catheter, a microcatheter, an osmotic pump, or an encapsulation device.

35. The article of claim 33 or claim 34, further comprising an additional therapeutic agent.
